SEQUENCE LISTING (8 sequences)

SEQ ID NO: 1 - protein, 120 aa, Artificial
  Synthetic VH
QIQLVQSGPELKKPGETVRISCKASGYTFTTSGMQWLQKMPGKGLKWIGWINTHFGEPKYAEDFKGRFAFSLETSASTAYLQISNLKNEDTATYFCAGAGYYGSRYFDVWGAGTTVTVSS

SEQ ID NO: 2 - protein, 8 aa, Artificial
  Synthetic VH-CDR1
GYTFTTSG

SEQ ID NO: 3 - protein, 8 aa, Artificial
  Synthetic VH-CDR2
INTHFGEP

SEQ ID NO: 4 - protein, 13 aa, Artificial
  Synthetic VH-CDR3
AGAGYYGSRYFDV

SEQ ID NO: 5 - protein, 107 aa, Artificial
  Synthetic VL
DIVMTQSQKFMSTSVGDRVSITCKASQNVGTAVAWYQQKPGQSPKLLIYSASNRYTGVPDRFTGSGSGTDFTLTISNMQSEDLADYFCQQYSSYPLTFGGGTKLEIK

SEQ ID NO: 6 - protein, 6 aa, Artificial
  Synthetic VL-CDR1
QNVGTA

SEQ ID NO: 7 - protein, 3 aa, Artificial
  Synthetic VL-CDR2
SAS

SEQ ID NO: 8 - protein, 9 aa, Artificial
  Synthetic VL-CDR3
QQYSSYPLT

(19) United States
(12) Patent Application Publication
(10) Pub. No.: US 2016/0185856 A1
(43) Pub. Date: Jun. 30, 2016

(54) Anti-Claudin 1 Antibodies and Uses Thereof

(71) Applicants: UNIVERSITÉ DE MONTPELLIER 1, Montpellier (FR); INSTITUT RÉGIONAL DU CANCER DE MONTPELLIER, Montpellier (FR); INSERM (INSTITUT NATIONAL DE LA SANTÉ ET DE LA RECHERCHE MÉDICALE), Paris (FR)

(72) Inventors: Marguerite DEL RIO, Montpellier Cedex 5 (FR); Nadia VEZZIO-VIE, Montpellier Cedex 5 (FR)

(73) Assignees: INSERM(Institut National de la Sante et de la Recherche Medicale), Paris (FR); Universite de Montpellier, Montpellier (FR); Institut Regional du Cancer de Montpellier, Montpellier (FR)

(21) Appl. No.: 14/909,362

(22) PCT Filed: Jul. 22, 2014
(86) PCT No.: PCT/EP2014/065679

§ 371 Date: Feb. 1, 2016

(30) Foreign Application Priority Data
Aug. 2, 2013 (EP) 13306115.0

(51) Int. Cl. C07K 16/28; G01N 33/574

(52) U.S. Cl. CPC C07K 16/28; G01N 33/57492; C07K 2317/565; C07K 2317/55; C07K 2317/54; C07K 2317/56; A61K 2039/505

(57) ABSTRACT

The present invention relates to anti-claudin 1 antibodies and their uses thereof. In particular, the present invention relates to an anti-Claudin 1 (CLDN1) antibody comprising n heavy chain variable region comprising SEQ ID NO:2 in the H-CDR1 region, SEQ ID NO:3 in the H-CDR2 region and SEQ ID NO:4 in the H-CDR3 region; and a light chain variable region comprising SEQ ID NO:6 in the L-CDR1 region, SEQ ID NO:7 in the L-CDR2 region and SEQ ID NO:8 in the L-CDR3 region.

### FIELD OF THE INVENTION

The present invention relates to anti-claudin 1 antibodies and their uses thereof.

### BACKGROUND OF THE INVENTION

The CLDNs are integral membrane proteins associated with tight junctions (TJs). TJs are located at the most apical region of the lateral membrane in epithelial cell and endothelial sheets. Their two major functions are a fence function that maintains cell polarity and a paracellular barrier function that regulates the diffusion of solutes (Tsukita and Furuse, 2006). CLDNs interact in two different ways: laterally in the plane of the membrane (heteromeric interactions) or head to head binding between adjacent cells (heterotypic interactions). They can form a complex with occludin and/or JAMs. They have a short intracellular N-terminal domain, four transmembrane domains, two extracellular loops and an intracellular C-terminal domain containing a phosphorylation site and a PDZ-domain-binding motif that allows claudins to interact directly with cytoplasmic scaffold proteins, such as the TJ-associated proteins MUPP1, PATJ, ZO-1, ZO-2 and ZO-3, and MAGUKs (Lal-Nag and Morin, 2009). These proteins might function as adaptors at the cytoplasmic surface of tight-junction strands to recruit other proteins including cytoskeletal and signalling molecules (Tsukita et al., 2001). A number of other cytosolic and nuclear proteins which includes regulatory proteins Rab3b, Rab13, tumor suppressors like PTEN, transcription factors like ZONAB, and HuASH1 have also been shown to interact directly or indirectly with tight junction complex (Singh et al., 2010). CLDN1 belongs to the claudin family of proteins which consists of 24 members of closely related transmembrane proteins. CLDN1 is an emerging therapeutic target in colorectal cancer or even for the treatment of infectious diseases such as HCV. Thus several anti-CLDN1 antibodies have been described in the prior art (WO2010034812, EP 1167 389 or in U.S. Pat. No. 6,627,439).

### SUMMARY OF THE INVENTION

The present invention relates to anti-claudin 1 antibodies and their uses thereof. In particular, the present invention relates to an anti-Claudin 1 (CLDN1) antibody comprising n heavy chain variable region comprising SEQ ID NO:2 in the H-CDR1 region, SEQ ID NO:3 in the H-CDR2 region and SEQ ID NO:4 in the H-CDR3 region; and a light chain variable region comprising SEQ ID NO:6 in the L-CDR1 region, SEQ ID NO:7 in the L-CDR2 region and SEQ ID NO:8 in the L-CDR3 region.

### DETAILED DESCRIPTION OF THE INVENTION

### Definitions

The term “Claudin-1” or “CLDN1” has its general meaning in the art and refers to the integral membrane protein associated with tight junction claudin-1. The CLDN1 has been first identified as a 22-kD polypeptide from isolated chicken liver junction fractions and cDNAs encoding their mouse homologues were cloned (Furuse et al., 1998). Human cDNA of CLDN1 (aliase=SEMP1) was cloned and sequenced (Swisshelm et al., 1999). It contains four exons including 636 nucleotides. The translation gives a product of 211 amino acid residues. CLDN1 has a tetraspan membrane topology with four transmembrane regions. Intracellularly, CLDN1 exhibits a 7 N-terminal amino acids, a 12 loop amino acids and a 27 C-terminal amino acids. The extracellular loop (ECL) 1 consists of 53 amino acids with two conserved cysteines. The ECL2 has 27 amino acids, The term “human Claudin-1 or human CLDN1” refers to a protein having the sequence shown in NCBI Accession Number NP_066924, or any naturally occurring variants commonly found in HCV permissive human populations. The term “extracellular domain” or “ectodomain” of Claudin-1 refers to the region of the Claudin-1 sequence that extends into the extracellular space (i.e., the space outside a cell).

The term “anti-CLDN1 antibody” refers to an antibody directed against CLDN1.

According to the present invention, “antibody” or “immunoglobulin” have the same meaning, and will be used equally in the present invention. The term “antibody” as used herein refers to immunoglobulin molecules and immunologically active portions of immunoglobulin molecules, i.e., molecules that contain an antigen binding site that immunospecifically binds an antigen. As such, the term antibody encompasses not only whole antibody molecules, but also antibody fragments as well as variants (including derivatives) of antibodies and antibody fragments. In natural antibodies, two heavy chains are linked to each other by disulfide bonds and each heavy chain is linked to a light chain by a disulfide bond. There are two types of light chain, lambda (l) and kappa (k). There are five main heavy chain classes (or isotypes) which determine the functional activity of an antibody molecule: IgM, IgD, IgG, IgA and IgE. Each chain contains distinct sequence domains. The light chain includes two domains, a variable domain (VL) and a constant domain (CL). The heavy chain includes four domains, a variable domain (VH) and three constant domains (CH1, CH2 and CH3, collectively referred to as CH). The variable regions of both light (VL) and heavy (VH) chains determine binding recognition and specificity to the antigen. The constant region domains of the light (CL) and heavy (CH) chains confer important biological properties such as antibody chain association, secretion, trans-placental mobility, complement binding, and binding to Fc receptors (FcR). The Fv fragment is the N-terminal part of the Fab fragment of an immunoglobulin and consists of the variable portions of one light chain and one heavy chain. The specificity of the antibody resides in the structural complementarity between the antibody combining site and the antigenic determinant. Antibody combining sites are made up of residues that are primarily from the hypervariable or complementarity determining regions (CDRs). Occasionally, residues from nonhypervariable or framework regions (FR) influence the overall domain structure and hence the combining site. Complementarity Determining Regions or CDRs refer to amino acid sequences which together define the binding affinity and specificity of the natural Fv region of a native immunoglobulin binding site. The light and heavy chains of an immunoglobulin each have three CDRs, designated L-CDR1, L-CDR2, L-CDR3 and H-CDR1, H-CDR2, H-CDR3, respectively. An antigen-binding site, therefore, includes six CDRs, comprising the CDR set from each of a heavy and a light chain V region. Framework Regions (FRs) refer to amino acid sequences interposed between CDRs.

The term “chimeric antibody” refers to an antibody which comprises a VH domain and a VL domain of an antibody derived the 6F6C3 antibody, and a CH domain and a CL domain of a human antibody.

According to the invention, the term “humanized antibody” refers to an antibody having variable region framework and constant regions from a human antibody but retains the CDRs of the 6F6C3 antibody.

The term “Fab” denotes an antibody fragment having a molecular weight of about 50,000 and antigen binding activity, in which about a half of the N-terminal side of H chain and the entire L chain, among fragments obtained by treating IgG with a protease, papaine, are bound together through a disulfide bond.

The term “F(ab′)2” refers to an antibody fragment having a molecular weight of about 100,000 and antigen binding activity, which is slightly larger than the Fab bound via a disulfide bond of the hinge region, among fragments obtained by treating IgG with a protease, pepsin.

The term “Fab′” refers to an antibody fragment having a molecular weight of about 50,000 and antigen binding activity, which is obtained by cutting a disulfide bond of the hinge region of the F(ab′)2.

A single chain Fv (“scFv”) polypeptide is a covalently linked VH::VL heterodimer which is usually expressed from a gene fusion including VH and VL encoding genes linked by a peptide-encoding linker. “dsFv” is a VH::VL heterodimer stabilised by a disulfide bond. Divalent and multivalent antibody fragments can form either spontaneously by association of monovalent scFvs, or can be generated by coupling monovalent scFvs by a peptide linker, such as divalent sc(Fv)2.

The term “diabodies” refers to small antibody fragments with two antigen-binding sites, which fragments comprise a heavy-chain variable domain (VH) connected to a light-chain variable domain (VL) in the same polypeptide chain (VH-VL). By using a linker that is too short to allow pairing between the two domains on the same chain, the domains are forced to pair with the complementary domains of another chain and create two antigen-binding sites.

By “purified” and “isolated” it is meant, when referring to an antibody according to the invention or to a nucleotide sequence, that the indicated molecule is present in the substantial absence of other biological macromolecules of the same type. The term “purified” as used herein preferably means at least 75% by weight, more preferably at least 85% by weight, more preferably still at least 95% by weight, and most preferably at least 98% by weight, of biological macromolecules of the same type are present. An “isolated” nucleic acid molecule which encodes a particular polypeptide refers to a nucleic acid molecule which is substantially free of other nucleic acid molecules that do not encode the polypeptide; however, the molecule may include some additional bases or moieties which do not deleteriously affect the basic characteristics of the composition.

### Antibodies of the Invention:

The present invention provides for isolated anti-CLDN1 antibodies or fragments thereof. In particular, the inventors have raised a murine anti-CLDN1 antibody (6F6C3) producing hybridoma. The inventors have cloned and characterized the variable domain of the light and heavy chains of said mAb 6F6C3, and thus determined the complementary determining regions (CDRs) domain of said antibody as described in Table 1:

TABLE 1
VH, VL and CDR domains of mAb6F6C3

| MAb | |
|---|---|
| 6F6C3 | |
| domains | Sequence |
| VH | QIQLVQSGPELKKPGETVRISCKASGYTFTTSGMQWLQKM |
| | PGKGLKWIGWINTHFGEPKYAEDFKGRFAFSLETSASTAY |
| | LQISNLKNEDTATYFCAGAGYYGSRYFDVWGAGTTVTVSS |
| | (SEQ ID NO: 1) |
| VH CDR1 | GYTFTTSG (SEQ ID NO: 2) |
| VH CDR2 | INTHFGEP (SEQ ID NO: 3) |
| VH CDR3 | AGAGYYGSRYFDV (SEQ ID NO: 4) |
| VL | DIVMTQSQKFMSTSVGDRVSITCKASQNVGTAVAWYQQKP |
| | GQSPKLLIYSASNRYTGVPDRFTGSGSGTDFTLTISNMQS |
| | EDLADYFCQQYSSYPLTFGGGTKLEIK (SEQ ID NO: |
| | 5) |
| VL CDR1 | QNVGTA (SEQ ID NO: 6) |
| VL CDR2 | SAS (SEQ ID NO: 7) |
| VL CDR3 | QQYSSYPLT (SEQ ID NO: 8) |

Therefore, the invention relates to a monoclonal antibody having specificity for CLDN1, comprising a heavy chain wherein the variable domain comprises at least one CDR having a sequence selected from the group consisting of SEQ ID NO:2 for H-CDR1, SEQ ID NO:3 for H-CDR2 and SEQ ID NO:4 for H-CDR3.

The invention also relates to a monoclonal antibody having specificity for CLDN1, comprising a light chain wherein the variable domain comprises at least one CDR having a sequence selected from the group consisting of SEQ ID NO:6 for L-CDR1, SEQ ID NO:7 for L-CDR2 and SEQ ID NO:8 for L-CDR3.

The monoclonal antibody of the invention, may comprise a heavy chain wherein the variable domain comprises at least one CDR having a sequence selected from the group consisting of SEQ ID NO:2 for H-CDR1, SEQ ID NO:3 for H-CDR2 and SEQ ID NO:4 for H-CDR3 and a light chain wherein the variable domain comprises at least one CDR having a sequence selected from the group consisting of SEQ ID NO:6 for L-CDR1, SEQ ID NO:7 for L-CDR2 and SEQ ID NO:8 for L-CDR3.

In particular, the invention provides an anti-CLDN1 monoclonal antibody comprising:
an heavy chain variable region comprising SEQ ID NO:2 in the H-CDR1 region, SEQ ID NO:3 in the H-CDR2 region and SEQ ID NO:4 in the H-CDR3 region; and
a light chain variable region comprising SEQ ID NO:6 in the L-CDR1 region, SEQ ID NO:7 in the L-CDR2 region and SEQ ID NO:8 in the L-CDR3 region.

In a particular embodiment, the heavy chain variable region of said antibody has the amino acid sequence set forth as SEQ ID NO: 1 and/or the light chain variable region has the amino acid sequence set forth as SEQ ID NO: 5.

In another embodiment, the monoclonal antibody of the invention is a chimeric antibody, preferably a chimeric mouse/human antibody. In particular, said mouse/human chimeric antibody may comprise the variable domains of 6F6C3 antibody as defined above.

In another embodiment, the monoclonal of the invention is a humanized antibody. In particular, in said humanized antibody, the variable domain comprises human acceptor frameworks regions, and optionally human constant domain where present, and non-human donor CDRs, such as mouse CDRs as defined above.

The invention further provides anti-CLDN1 fragments directed against CLDN1 of said antibodies which include but are not limited to Fv, Fab, F(ab′)2, Fab′, dsFv, scFv, sc(Fv)2 and diabodies.

In another aspect, the invention relates to a polypeptide which has a sequence selected from the group consisting of SEQ ID NO: 1, SEQ ID NO:2, SEQ ID NO:3, SEQ ID NO:4, SEQ ID NO:5; SEQ ID NO: 6; SEQ ID NO:7 and SEQ ID NO:8.

Methods of Producing Antibodies of the Invention:

Anti-CLDN1 antibodies of the invention may be produced by any technique known in the art, such as, without limitation, any chemical, biological, genetic or enzymatic technique, either alone or in combination.

Knowing the amino acid sequence of the desired sequence, one skilled in the art can readily produce said antibodies, by standard techniques for production of polypeptides. For instance, they can be synthesized using well-known solid phase method, preferably using a commercially available peptide synthesis apparatus (such as that made by Applied Biosystems, Foster City, Calif.) and following the manufacturer's instructions. Alternatively, antibodies of the invention can be synthesized by recombinant DNA techniques well-known in the art. For example, antibodies can be obtained as DNA expression products after incorporation of DNA sequences encoding the antibodies into expression vectors and introduction of such vectors into suitable eukaryotic or prokaryotic hosts that will express the desired antibodies, from which they can be later isolated using well-known techniques.

Accordingly, a further object of the invention relates to a nucleic acid sequence encoding an antibody according to the invention. More particularly the nucleic acid sequence encodes a heavy chain or a light chain of an antibody of the invention.

Typically, said nucleic acid is a DNA or RNA molecule, which may be included in any suitable vector, such as a plasmid, cosmid, episome, artificial chromosome, phage or a viral vector.

The terms “vector”, “cloning vector” and “expression vector” mean the vehicle by which a DNA or RNA sequence (e.g. a foreign gene) can be introduced into a host cell, so as to transform the host and promote expression (e.g. transcription and translation) of the introduced sequence.

So, a further object of the invention relates to a vector comprising a nucleic acid of the invention.

Such vectors may comprise regulatory elements, such as a promoter, enhancer, terminator and the like, to cause or direct expression of said antibody upon administration to a subject. Examples of promoters and enhancers used in the expression vector for animal cell include early promoter and enhancer of SV40, LTR promoter and enhancer of Moloney mouse leukemia virus, promoter and enhancer of immunoglobulin H chain and the like.

Any expression vector for animal cell can be used, so long as a gene encoding the human antibody C region can be inserted and expressed. Examples of plasmids include replicating plasmids comprising an origin of replication, or integrative plasmids, such as for instance pUC, pcDNA, pBR, and the like. Other examples of viral vector include adenoviral, retroviral, herpes virus and AAV vectors. Such recombinant viruses may be produced by techniques known in the art, such as by transfecting packaging cells or by transient transfection with helper plasmids or viruses.

A further object of the present invention relates to a host cell which has been transfected, infected or transformed by a nucleic acid and/or a vector according to the invention.

The term “transformation” means the introduction of a “foreign” (i.e. extrinsic or extracellular) gene, DNA or RNA sequence to a host cell, so that the host cell will express the introduced gene or sequence to produce a desired substance, typically a protein or enzyme coded by the introduced gene or sequence. A host cell that receives and expresses introduced DNA or RNA has been “transformed”.

The nucleic acids of the invention may be used to produce an antibody of the invention in a suitable expression system. The term “expression system” means a host cell and compatible vector under suitable conditions, e.g. for the expression of a protein coded for by foreign DNA carried by the vector and introduced to the host cell.

Common expression systems include E. coli host cells and plasmid vectors, insect host cells and Baculovirus vectors, and mammalian host cells and vectors. Other examples of host cells include, without limitation, prokaryotic cells (such as bacteria) and eukaryotic cells (such as yeast cells, mammalian cells, insect cells, plant cells, etc.). Specific examples include E. coli, Kluyveromyces or Saccharomyces yeasts, mammalian cell lines (e.g., Vero cells, CHO cells, 3T3 cells, COS cells, etc.) as well as primary or established mammalian cell cultures (e.g., produced from lymphoblasts, fibroblasts, embryonic cells, epithelial cells, nervous cells, adipocytes, etc.). Examples also include mouse SP2/0-Ag14 cell (ATCC CRL1581), mouse P3X63-Ag8.653 cell (ATCC CRL1580), CHO cell in which a dihydrofolate reductase gene (hereinafter referred to as “DHFR gene”) is defective, rat YB2/3HL.P2.G11.16Ag.20 cell (ATCC CRL1662, hereinafter referred to as “YB2/0 cell”), and the like.

The present invention also relates to a method of producing a recombinant host cell expressing an antibody according to the invention, said method comprising the steps of: (i) introducing in vitro or ex vivo a recombinant nucleic acid or a vector as described above into a competent host cell, (ii) culturing in vitro or ex vivo the recombinant host cell obtained and (iii), optionally, selecting the cells which express and/or secrete said antibody. Such recombinant host cells can be used for the production of antibodies of the invention.

In another particular embodiment, the method comprises the steps of (i) culturing the hybridoma 6F6C3 under conditions suitable to allow expression of 6F6C3 antibody; and (ii) recovering the expressed antibody.

Antibodies of the invention are suitably separated from the culture medium by conventional immunoglobulin purification procedures such as, for example, protein A-Sepharose, hydroxylapatite chromatography, gel electrophoresis, dialysis, or affinity chromatography.

In a particular embodiment, the human chimeric antibody of the present invention can be produced by obtaining nucleic sequences encoding VL and VH domains as previously described, constructing a human chimeric antibody expression vector by inserting them into an expression vector for animal cell having genes encoding human antibody CH and human antibody CL, and expressing the coding sequence by introducing the expression vector into an animal cell.

As the CH domain of a human chimeric antibody, it may be any region which belongs to human immunoglobulin, but those of IgG class are suitable and any one of subclasses belonging to IgG class, such as IgG1, IgG2, IgG3 and IgG4, can also be used. Also, as the CL of a human chimeric antibody, it may be any region which belongs to Ig, and those of kappa class or lambda class can be used.

Methods for producing chimeric antibodies involve conventional recombinant DNA and gene transfection techniques are well known in the art (See patent documents U.S. Pat. No. 5,202,238; and U.S. Pat. No. 5,204,244).

The humanized antibody of the present invention may be produced by obtaining nucleic acid sequences encoding CDR domains, as previously described, constructing a humanized antibody expression vector by inserting them into an expression vector for animal cell having genes encoding (i) a heavy chain constant region identical to that of a human antibody and (ii) a light chain constant region identical to that of a human antibody, and expressing the genes by introducing the expression vector into an animal cell.

The humanized antibody expression vector may be either of a type in which a gene encoding an antibody heavy chain and a gene encoding an antibody light chain exists on separate vectors or of a type in which both genes exist on the same vector (tandem type). In respect of easiness of construction of a humanized antibody expression vector, easiness of introduction into animal cells, and balance between the expression levels of antibody H and L chains in animal cells, humanized antibody expression vector of the tandem type is preferred. Examples of tandem type humanized antibody expression vector include pKANTEX93 (WO 97/10354), pEE18 and the like.

Methods for producing humanized antibodies based on conventional recombinant DNA and gene transfection techniques are well known in the art (See, e. g., Riechmann L. et al. 1988; Neuberger M S. et al. 1985). Antibodies can be humanized using a variety of techniques known in the art including, for example, CDR-grafting (EP 239,400; PCT publication WO91/09967; U.S. Pat. Nos. 5,225,539; 5,530,101; and 5,585,089), veneering or resurfacing (EP 592,106; EP 519,596; Padlan E A (1991); Studnicka G M et al. (1994); Roguska M A. et al. (1994)), and chain shuffling (U.S. Pat. No. 5,565,332). The general recombinant DNA technology for preparation of such antibodies is also known (see European Patent Application EP 125023 and International Patent Application WO 96/02576).

The Fab of the present invention can be obtained by treating an antibody which specifically reacts with CLDN1 with a protease, papaine. Also, the Fab can be produced by inserting DNA encoding Fab of the antibody into a vector for prokaryotic expression system, or for eukaryotic expression system, and introducing the vector into a prokaryote or eucaryote (as appropriate) to express the Fab.

The F(ab′)2 of the present invention can be obtained treating an antibody which specifically reacts with CLDN1 with a protease, pepsin. Also, the F(ab′)2 can be produced by binding Fab′ described below via a thioether bond or a disulfide bond.

The Fab′ of the present invention can be obtained treating F(ab′)2 which specifically reacts with CLDN1 with a reducing agent, dithiothreitol. Also, the Fab′ can be produced by inserting DNA encoding Fab′ fragment of the antibody into an expression vector for prokaryote, or an expression vector for eukaryote, and introducing the vector into a prokaryote or eukaryote (as appropriate) to perform its expression.

The scFv of the present invention can be produced by obtaining cDNA encoding the VH and VL domains as previously described, constructing DNA encoding scFv, inserting the DNA into an expression vector for prokaryote, or an expression vector for eukaryote, and then introducing the expression vector into a prokaryote or eukaryote (as appropriate) to express the scFv. To generate a humanized scFv fragment, a well known technology called CDR grafting may be used, which involves selecting the complementary determining regions (CDRs) from a donor scFv fragment, and grafting them onto a human scFv fragment framework of known three dimensional structure (see, e. g., W098/45322; WO 87/02671; U.S. Pat. No. 5,859,205; U.S. Pat. No. 5,585,089; U.S. Pat. No. 4,816,567; EP0173494).

Amino acid sequence modification(s) of the antibodies described herein are contemplated. For example, it may be desirable to improve the binding affinity and/or other biological properties of the antibody. It is known that when a humanized antibody is produced by simply grafting only CDRs in VH and VL of an antibody derived from a non-human animal in FRs of the VH and VL of a human antibody, the antigen binding activity is reduced in comparison with that of the original antibody derived from a non-human animal. It is considered that several amino acid residues of the VH and VL of the non-human antibody, not only in CDRs but also in FRs, are directly or indirectly associated with the antigen binding activity. Hence, substitution of these amino acid residues with different amino acid residues derived from FRs of the VH and VL of the human antibody would reduce of the binding activity. In order to resolve the problem, in antibodies grafted with human CDR, attempts have to be made to identify, among amino acid sequences of the FR of the VH and VL of human antibodies, an amino acid residue which is directly associated with binding to the antibody, or which interacts with an amino acid residue of CDR, or which maintains the three-dimensional structure of the antibody and which is directly associated with binding to the antigen. The reduced antigen binding activity could be increased by replacing the identified amino acids with amino acid residues of the original antibody derived from a non-human animal.

Modifications and changes may be made in the structure of the antibodies of the present invention, and in the DNA sequences encoding them, and still obtain a functional molecule that encodes an antibody with desirable characteristics.

In making the changes in the amino sequences, the hydropathic index of amino acids may be considered. The importance of the hydropathic amino acid index in conferring interactive biologic function on a protein is generally understood in the art. It is accepted that the relative hydropathic character of the amino acid contributes to the secondary structure of the resultant protein, which in turn defines the interaction of the protein with other molecules, for example, enzymes, substrates, receptors, DNA, antibodies, antigens, and the like. Each amino acid has been assigned a hydropathic index on the basis of their hydrophobicity and charge characteristics these are: isoleucine (+4.5); valine (+4.2); leucine (+3.8); phenylalanine (+2.8); cysteine/cystine (+2.5); methionine (+1.9); alanine (+1.8); glycine (−0.4); threonine (−0.7); serine (−0.8); tryptophane (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5).

A further object of the present invention also encompasses function-conservative variants of the antibodies of the present invention.

“Function-conservative variants” are those in which a given amino acid residue in a protein or enzyme has been changed without altering the overall conformation and function of the polypeptide, including, but not limited to, replacement of an amino acid with one having similar properties (such as, for example, polarity, hydrogen bonding potential, acidic, basic, hydrophobic, aromatic, and the like). Amino acids other than those indicated as conserved may differ in a protein so that the percent protein or amino acid sequence similarity between any two proteins of similar function may vary and may be, for example, from 70% to 99% as determined according to an alignment scheme such as by the Cluster Method, wherein similarity is based on the MEGALIGN algorithm. A “function-conservative variant” also includes a polypeptide which has at least 60% amino acid identity as determined by BLAST or FASTA algorithms, preferably at least 75%, more preferably at least 85%, still preferably at least 90%, and even more preferably at least 95%, and which has the same or substantially similar properties or functions as the native or parent protein to which it is compared.

Two amino acid sequences are “substantially homologous” or “substantially similar” when greater than 80%, preferably greater than 85%, preferably greater than 90% of the amino acids are identical, or greater than about 90%, preferably greater than 95%, are similar (functionally identical) over the whole length of the shorter sequence. Preferably, the similar or homologous sequences are identified by alignment using, for example, the GCG (Genetics Computer Group, Program Manual for the GCG Package, Version 7, Madison, Wis.) pileup program, or any of sequence comparison algorithms such as BLAST, FASTA, etc.

For example, certain amino acids may be substituted by other amino acids in a protein structure without appreciable loss of activity. Since the interactive capacity and nature of a protein define the protein's biological functional activity, certain amino acid substitutions can be made in a protein sequence, and, of course, in its DNA encoding sequence, while nevertheless obtaining a protein with like properties. It is thus contemplated that various changes may be made in the antibodies sequences of the invention, or corresponding DNA sequences which encode said antibodies, without appreciable loss of their biological activity.

It is known in the art that certain amino acids may be substituted by other amino acids having a similar hydropathic index or score and still result in a protein with similar biological activity, i.e. still obtain a biological functionally equivalent protein.

As outlined above, amino acid substitutions are generally therefore based on the relative similarity of the amino acid side-chain substituents, for example, their hydrophobicity, hydrophilicity, charge, size, and the like. Exemplary substitutions which take various of the foregoing characteristics into consideration are well known to those of skill in the art and include: arginine and lysine; glutamate and aspartate; serine and threonine; glutamine and asparagine; and valine, leucine and isoleucine.

Accordingly, the invention also provides an antibody comprising a heavy chain wherein the variable domain comprises:
a H-CDR1 having at least 90% or 95% identity with sequence set forth as SEQ ID NO: 2,
a H-CDR2 having at least 90% or 95% identity with sequence set forth as SEQ ID NO: 3,
a H-CDR3 having at least 90% or 95% identity with sequence set forth as SEQ ID NO: 4,
a L-CDR1 having at least 90% or 95% identity with sequence set forth as SEQ ID NO: 6,
a L-CDR2 having at least 90% or 95% identity with sequence set forth as SEQ ID NO: 7,
a L-CDR3 having at least 90% or 95% identity with sequence set forth as SEQ ID NO: 8, and
that specifically binds to CLDN1 with substantially the same affinity as an antibody comprising a heavy chain wherein the variable domain comprises SEQ ID NO: 2 for H-CDR1, SEQ ID NO: 3 for H-CDR2 and SEQ ID NO: 4 for H-CDR3 and a light chain wherein the variable domain comprises SEQ ID NO: 6 for L-CDR1, SEQ ID NO: 7 for L-CDR2 and SEQ ID NO: 8 for L-CDR3, and more preferably with substantially the same affinity as the murine anti-CLDN1 antibody 6F6C3.

Said antibodies may be assayed for specific binding by any method known in the art. Many different competitive binding assay format(s) can be used for epitope binning. The immunoassays which can be used include, but are not limited to, competitive assay systems using techniques such western blots, radioimmunoassays, ELISA, “sandwich” immunoassays, immunoprecipitation assays, precipitin assays, gel diffusion precipitin assays, immunoradiometric assays, fluorescent immunoassays, protein A immunoassays, and complement-fixation assays. Such assays are routine and well known in the art (see, e.g., Ausubel et al., eds, 1994 Current Protocols in Molecular Biology, Vol. 1, John Wiley & sons, Inc., New York). For example, the BIACORE® (GE Healthcare, Piscaataway, N.J.) is one of a variety of surface plasmon resonance assay formats that are routinely used to epitope bin panels of monoclonal antibodies. Additionally, routine cross-blocking assays such as those described in Antibodies, A Laboratory Manual, Cold Spring Harbor Laboratory, Ed Harlow and David Lane, 1988, can be performed.

Engineered antibodies of the invention include those in which modifications have been made to framework residues within VH and/or VL, e.g. to improve the properties of the antibody. Typically such framework modifications are made to decrease the immunogenicity of the antibody. For example, one approach is to “backmutate” one or more framework residues to the corresponding germline sequence. More specifically, an antibody that has undergone somatic mutation may contain framework residues that differ from the germline sequence from which the antibody is derived. Such residues can be identified by comparing the antibody framework sequences to the germline sequences from which the antibody is derived. To return the framework region sequences to their germline configuration, the somatic mutations can be “backmutated” to the germline sequence by, for example, site-directed mutagenesis or PCR-mediated mutagenesis. Such “backmutated” antibodies are also intended to be encompassed by the invention. Another type of framework modification involves mutating one or more residues within the framework region, or even within one or more CDR regions, to remove T cell-epitopes to thereby reduce the potential immunogenicity of the antibody. This approach is also referred to as “deimmunization” and is described in further detail in U.S. Patent Publication No. 20030153043 by Carr et al.

In addition or alternative to modifications made within the framework or CDR regions, antibodies of the invention may be engineered to include modifications within the Fc region, typically to alter one or more functional properties of the antibody, such as serum half-life, complement fixation, Fc receptor binding, and/or antigen-dependent cellular cytotoxicity. Furthermore, an antibody of the invention may be chemically modified (e.g., one or more chemical moieties can be attached to the antibody) or be modified to alter its glycosylation, again to alter one or more functional properties of the antibody. Each of these embodiments is described in further detail below. The numbering of residues in the Fc region is that of the EU index of Kabat.

In one embodiment, the hinge region of CH1 is modified such that the number of cysteine residues in the hinge region is altered, e.g., increased or decreased. This approach is described further in U.S. Pat. No. 5,677,425 by Bodmer et al. The number of cysteine residues in the hinge region of CH1 is altered to, for example, facilitate assembly of the light and heavy chains or to increase or decrease the stability of the antibody.

In another embodiment, the Fc hinge region of an antibody is mutated to decrease the biological half-life of the antibody. More specifically, one or more amino acid mutations are introduced into the CH2-CH3 domain interface region of the Fc-hinge fragment such that the antibody has impaired Staphylococcyl protein A (SpA) binding relative to native Fc-hinge domain SpA binding. This approach is described in further detail in U.S. Pat. No. 6,165,745 by Ward et al.

In another embodiment, the antibody is modified to increase its biological half-life. Various approaches are possible. For example, one or more of the following mutations can be introduced: T252L, T254S, T256F, as described in U.S. Pat. No. 6,277,375 by Ward. Alternatively, to increase the biological half life, the antibody can be altered within the CH1 or CL region to contain a salvage receptor binding epitope taken from two loops of a CH2 domain of an Fc region of an IgG, as described in U.S. Pat. Nos. 5,869,046 and 6,121,022 by Presta et al.

In yet other embodiments, the Fc region is altered by replacing at least one amino acid residue with a different amino acid residue to alter the effector functions of the antibody. For example, one or more amino acids can be replaced with a different amino acid residue such that the antibody has an altered affinity for an effector ligand but retains the antigen-binding ability of the parent antibody. The effector ligand to which affinity is altered can be, for example, an Fc receptor or the C1 component of complement. This approach is described in further detail in U.S. Pat. Nos. 5,624,821 and 5,648,260, both by Winter et al.

In another embodiment, one or more amino acids selected from amino acid residues can be replaced with a different amino acid residue such that the antibody has altered C1 q binding and/or reduced or abolished complement dependent cytotoxicity (CDC). This approach is described in further detail in U.S. Pat. No. 6,194,551 by ldusogie et al.

In another embodiment, one or more amino acid residues are altered to thereby alter the ability of the antibody to fix complement. This approach is described further in PCT Publication WO 94/29351 by Bodmer et al.

In yet another embodiment, the Fc region is modified to increase the ability of the antibody to mediate antibody dependent cellular cytotoxicity (ADCC) and/or to increase the affinity of the antibody for an Fc receptor by modifying one or more amino acids. This approach is described further in PCT Publication WO 00/42072 by Presta. Moreover, the binding sites on human IgGI for FcγRI, FcγRII, FcγRIII and FcRn have been mapped and variants with improved binding have been described (see Shields, R. L. et al., 2001 J. Biol. Chen. 276:6591-6604, WO2010106180).

In still another embodiment, the glycosylation of an antibody is modified. For example, an aglycoslated antibody can be made (i.e., the antibody lacks glycosylation). Glycosylation can be altered to, for example, increase the affinity of the antibody for the antigen. Such carbohydrate modifications can be accomplished by, for example, altering one or more sites of glycosylation within the antibody sequence. For example, one or more amino acid substitutions can be made that result in elimination of one or more variable region framework glycosylation sites to thereby eliminate glycosylation at that site. Such aglycosylation may increase the affinity of the antibody for antigen. Such an approach is described in further detail in U.S. Pat. Nos. 5,714,350 and 6,350,861 by Co et al.

Additionally or alternatively, an antibody can be made that has an altered type of glycosylation, such as a hypofucosylated or non-fucosylated antibody having reduced amounts of or no fucosyl residues or an antibody having increased bisecting GlcNac structures. Such altered glycosylation patterns have been demonstrated to increase the ADCC ability of antibodies. Such carbohydrate modifications can be accomplished by, for example, expressing the antibody in a host cell with altered glycosylation machinery. Cells with altered glycosylation machinery have been described in the art and can be used as host cells in which to express recombinant antibodies of the invention to thereby produce an antibody with altered glycosylation. For example, EP 1,176,195 by Hang et al. describes a cell line with a functionally disrupted FUT8 gene, which encodes a fucosyl transferase, such that antibodies expressed in such a cell line exhibit hypofucosylation or are devoid of fucosyl residues. Therefore, in one embodiment, the antibodies of the invention may be produced by recombinant expression in a cell line which exhibit hypofucosylation or non-fucosylation pattern, for example, a mammalian cell line with deficient expression of the FUT8 gene encoding fucosyltransferase. PCT Publication WO 03/035835 by Presta describes a variant CHO cell line, Lec13 cells, with reduced ability to attach fucose to Asn(297)-linked carbohydrates, also resulting in hypofucosylation of antibodies expressed in that host cell (see also Shields, R. L. et al., 2002 J. Biol. Chem. 277:26733-26740). PCT Publication WO 99/54342 by Umana et al. describes cell lines engineered to express glycoprotein-modifying glycosyl transferases (e.g., beta(1,4)-N acetylglucosaminyltransferase III (GnTIII)) such that antibodies expressed in the engineered cell lines exhibit increased bisecting GlcNac structures which results in increased ADCC activity of the antibodies (see also Umana et al., 1999 Nat. Biotech. 17:176-180). Eureka Therapeutics further describes genetically engineered CHO mammalian cells capable of producing antibodies with altered mammalian glycosylation pattern devoid of fucosyl residues. Alternatively, the antibodies of the invention can be produced in yeasts or filamentous fungi engineered for mammalian-like glycosylation pattern and capable of producing antibodies lacking fucose as glycosylation pattern (see for example EP1297172B1).

Another modification of the antibodies herein that is contemplated by the invention is pegylation. An antibody can be pegylated to, for example, increase the biological (e.g., serum) half-life of the antibody. To pegylate an antibody, the antibody, or fragment thereof, typically is reacted with polyethylene glycol (PEG), such as a reactive ester or aldehyde derivative of PEG, under conditions in which one or more PEG groups become attached to the antibody or antibody fragment. The pegylation can be carried out by an acylation reaction or an alkylation reaction with a reactive PEG molecule (or an analogous reactive water-soluble polymer). As used herein, the term “polyethylene glycol” is intended to encompass any of the forms of PEG that have been used to derivatize other proteins, such as mono (C1-C10) alkoxy- or aryloxy-polyethylene glycol or polyethylene glycol-maleimide. In certain embodiments, the antibody to be pegylated is an aglycosylated antibody. Methods for pegylating proteins are known in the art and can be applied to the antibodies of the invention. See for example, EP O 154 316 by Nishimura et al. and EP 0 401 384 by Ishikawa et al.

Another modification of the antibodies that is contemplated by the invention is a conjugate or a protein fusion of at least the antigen-binding region of the antibody of the invention to serum protein, such as human serum albumin or a fragment thereof to increase half-life of the resulting molecule. Such approach is for example described in Ballance et al. EP0322094.

Another possibility is a fusion of at least the antigen-binding region of the antibody of the invention to proteins capable of binding to serum proteins, such human serum albumin to increase half life of the resulting molecule. Such approach is for example described in Nygren et al., EP 0 486 525.

Immunoconjugates:

An antibody of the invention can be conjugated with a detectable label to form an anti-CLDN1 immunoconjugate. Suitable detectable labels include, for example, a radioisotope, a fluorescent label, a chemiluminescent label, an enzyme label, a bioluminescent label or colloidal gold. Methods of making and detecting such detectably-labeled immunoconjugates are well-known to those of ordinary skill in the art, and are described in more detail below.

The detectable label can be a radioisotope that is detected by autoradiography. Isotopes that are particularly useful for the purpose of the present invention are ³H, ¹²⁵I, ¹³¹I, ³⁵S and ¹⁴C.

Anti-CLDN1 immunoconjugates can also be labeled with a fluorescent compound. The presence of a fluorescently-labeled antibody is determined by exposing the immunoconjugate to light of the proper wavelength and detecting the resultant fluorescence. Fluorescent labeling compounds include fluorescein isothiocyanate, rhodamine, phycoerytherin, phycocyanin, allophycocyanin, o-phthaldehyde and fluorescamine.

Alternatively, anti-CLDN1 immunoconjugates can be detectably labeled by coupling an antibody to a chemiluminescent compound. The presence of the chemiluminescent-tagged immunoconjugate is determined by detecting the presence of luminescence that arises during the course of a chemical reaction. Examples of chemiluminescent labeling compounds include luminol, isoluminol, an aromatic acridinium ester, an imidazole, an acridinium salt and an oxalate ester.

Similarly, a bioluminescent compound can be used to label anti-CLDN1 immunoconjugates of the present invention. Bioluminescence is a type of chemiluminescence found in biological systems in which a catalytic protein increases the efficiency of the chemiluminescent reaction. The presence of a bioluminescent protein is determined by detecting the presence of luminescence. Bioluminescent compounds that are useful for labeling include luciferin, luciferase and aequorin.

Alternatively, anti-CLDN1 immunoconjugates can be detectably labeled by linking an anti-CLDN1 monoclonal antibody to an enzyme. When the anti-CLDN1-enzyme conjugate is incubated in the presence of the appropriate substrate, the enzyme moiety reacts with the substrate to produce a chemical moiety which can be detected, for example, by spectrophotometric, fluorometric or visual means. Examples of enzymes that can be used to detectably label polyspecific immunoconjugates include β-galactosidase, glucose oxidase, peroxidase and alkaline phosphatase.

Those of skill in the art will know of other suitable labels which can be employed in accordance with the present invention. The binding of marker moieties to anti-CLDN1 monoclonal antibodies can be accomplished using standard techniques known to the art.

Moreover, the convenience and versatility of immunochemical detection can be enhanced by using anti-CLDN1 monoclonal antibodies that have been conjugated with avidin, streptavidin, and biotin.

In another aspect, the present invention provides an anti-CLDN1 monoclonal antibody-drug conjugate. An “anti-CLDN1 monoclonal antibody-drug conjugate” as used herein refers to an anti-CLDN1 monoclonal antibody according to the invention conjugated to a therapeutic agent. Such anti-CLDN1 monoclonal antibody-drug conjugates produce clinically beneficial effects on CLDN1-expressing cells when administered to a subject, such as, for example, a subject with a CLDN1-expressing cancer, typically when administered alone but also in combination with other therapeutic agents.

In typical embodiments, an anti-CLDN1 monoclonal antibody is conjugated to a cytotoxic agent, such that the resulting antibody-drug conjugate exerts a cytotoxic or cytostatic effect on a CLDN1-expressing cell (e.g., a CLDN1-expressing cancer cell) when taken up or internalized by the cell. Particularly suitable moieties for conjugation to antibodies are chemotherapeutic agents, prodrug converting enzymes, radioactive isotopes or compounds, or toxins. For example, an anti-CLDN1 monoclonal antibody can be conjugated to a cytotoxic agent such as a chemotherapeutic agent or a toxin (e.g., a cytostatic or cytocidal agent such as, for example, abrin, ricin A, pseudomonas exotoxin, or diphtheria toxin).

Useful classes of cytotoxic agents include, for example, antitubulin agents, auristatins, DNA minor groove binders, DNA replication inhibitors, alkylating agents (e.g., platinum complexes such as cis-platin, mono(platinum), bis(platinum) and tri-nuclear platinum complexes and -carboplatin), anthracyclines, antibiotics, antifolates, antimetabolites, chemotherapy sensitizers, duocarmycins, etoposides, fluorinated pyrimidines, ionophores, lexitropsins, nitrosoureas, platinols, pre-forming compounds, purine antimetabolites, puromycins, radiation sensitizers, steroids, taxanes, topoisomerase inhibitors, vinca alkaloids, or the like.

Individual cytotoxic agents include, for example, an androgen, anthramycin (AMC), asparaginase, 5-azacytidine, azathioprine, bleomycin, busulfan, buthionine sulfoximine, camptothecin, carboplatin, carmustine (BSNU), CC-1065 (Li et al., Cancer Res. 42:999-1004, 1982), chlorambucil, cisplatin, colchicine, cyclophosphamide, cytarabine, cytidine arabinoside, cytochalasin B, dacarbazine, dactinomycin (formerly actinomycin), daunorubicin, decarbazine, docetaxel, doxorubicin, an estrogen, 5-fluordeoxyuridine, etopside phosphate (VP-16), 5-fluorouracil, gramicidin D, hydroxyurea, idarubicin, ifosfamide, irinotecan, lomustine (CCNU), mechlorethamine, melphalan, 6-mercaptopurine, methotrexate, mithramycin, mitomycin C, mitoxantrone, nitroimidazole, paclitaxel, plicamycin, procarbizine, streptozotocin, tenoposide (VM-26), 6-thioguanine, thioTEPA, topotecan, vinblastine, vincristine, and vinorelbine.

Particularly suitable cytotoxic agents include, for example, dolastatins (e.g., auristatin E, AFP, MMAF, MMAE), DNA minor groove binders (e.g., enediynes and lexitropsins), duocarmycins, taxanes (e.g., paclitaxel and docetaxel), puromycins, vinca alkaloids, CC-1065, SN-38 (7-ethyl-10-hydroxy-camptothein), topotecan, morpholino-doxorubicin, rhizoxin, cyanomorpholino-doxorubicin, echinomycin, combretastatin, netropsin, epothilone A and B, estramustine, cryptophysins, cemadotin, maytansinoids, discodermolide, eleutherobin, and mitoxantrone.

In certain embodiments, a cytotoxic agent is a conventional chemotherapeutic such as, for example, doxorubicin, paclitaxel, melphalan, vinca alkaloids, methotrexate, mitomycin C or etoposide. In addition, potent agents such as CC-1065 analogues, calicheamicin, maytansine, analogues of dolastatin 10, rhizoxin, and palytoxin can be linked to an anti-CLDN1-expressing antibody.

In specific variations, the cytotoxic or cytostatic agent is auristatin E (also known in the art as dolastatin-10) or a derivative thereof. Typically, the auristatin E derivative is, e.g., an ester formed between auristatin E and a keto acid. For example, auristatin E can be reacted with paraacetyl benzoic acid or benzoylvaleric acid to produce AEB and AEVB, respectively. Other typical auristatin derivatives include AFP (dimethylvaline-valine-dolaisoleuine-dolaproine-phenylalanine-p-phenylenediamine), MMAF (dovaline-valine-dolaisoleunine-dolaproine-phenylalanine), and MAE (monomethyl auristatin E). The synthesis and structure of auristatin E and its derivatives are described in U.S. Patent Application Publication No. 20030083263; International Patent Publication Nos. WO 2002/088172 and WO 2004/010957; and U.S. Pat. Nos. 6,884,869; 6,323,315; 6,239,104; 6,034,065; 5,780,588; 5,665,860; 5,663,149; 5,635,483; 5,599,902; 5,554,725; 5,530,097; 5,521,284; 5,504,191; 5,410,024; 5,138,036; 5,076,973; 4,986,988; 4,978,744; 4,879,278; 4,816,444; and 4,486,414.

In other variations, the cytotoxic agent is a DNA minor groove binding agent. (See, e.g., U.S. Pat. No. 6,130,237.) For example, in certain embodiments, the minor groove binding agent is a CBI compound. In other embodiments, the minor groove binding agent is an enediyne (e.g., calicheamicin).

In certain embodiments, an antibody-drug conjugate comprises an anti-tubulin agent. Examples of anti-tubulin agents include, for example, taxanes (e.g., Taxol® (paclitaxel), Taxotere® (docetaxel)), T67 (Tularik), vinca alkyloids (e.g., vincristine, vinblastine, vindesine, and vinorelbine), and dolastatins (e.g., auristatin E, AFP, MMAF, MMAE, AEB, AEVB). Other antitubulin agents include, for example, baccatin derivatives, taxane analogs (e.g., epothilone A and B), nocodazole, colchicine and colcimid, estramustine, cryptophysins, cemadotin, maytansinoids, combretastatins, discodermolide, and eleutherobin. In some embodiments, the cytotoxic agent is a maytansinoid, another group of anti-tubulin agents. For example, in specific embodiments, the maytansinoid is maytansine or DM-1 (ImmunoGen, Inc.; see also Chari et al., Cancer Res. 52:127-131, 1992).

In other embodiments, the cytotoxic agent is an antimetabolite. The antimetabolite can be, for example, a purine antagonist (e.g., azothioprine or mycophenolate mofetil), a dihydrofolate reductase inhibitor (e.g., methotrexate), acyclovir, gangcyclovir, zidovudine, vidarabine, ribavarin, azidothymidine, cytidine arabino side, amantadine, dideoxyuridine, iododeoxyuridine, poscarnet, or trifluridine.

In other embodiments, an anti-CLDN1 monoclonal antibody is conjugated to a pro-drug converting enzyme. The pro-drug converting enzyme can be recombinantly fused to the antibody or chemically conjugated thereto using known methods. Exemplary pro-drug converting enzymes are carboxypeptidase G2, β-glucuronidase, penicillin-V-amidase, penicillin-G-amidase, β-lactamase, β-glucosidase, nitroreductase and carboxypeptidase A.

Techniques for conjugating molecule to antibodies, are well-known in the art (See, e.g., Arnon et al., “Monoclonal Antibodies For Immunotargeting Of Drugs In Cancer Therapy,” in Monoclonal Antibodies And Cancer Therapy (Reisfeld et al. eds., Alan R. Liss, Inc., 1985); Hellstrom et al., “Antibodies For Drug Delivery,” in Controlled Drug Delivery (Robinson et al. eds., Marcel Deiker, Inc., 2nd ed. 1987); Thorpe, “Antibody Carriers Of Cytotoxic Agents In Cancer Therapy: A Review,” in Monoclonal Antibodies '84: Biological And Clinical Applications (Pinchera et al. eds., 1985); “Analysis, Results, and Future Prospective of the Therapeutic Use of Radiolabeled Antibody In Cancer Therapy,” in Monoclonal Antibodies For Cancer Detection And Therapy (Baldwin et al. eds., Academic Press, 1985); and Thorpe et al., 1982, Immunol. Rev. 62:119-58. See also, e.g., PCT publication WO 89/12624.) Typically, the nucleic acid molecule is covalently attached to lysines or cysteines on the antibody, through N-hydroxysuccinimide ester or maleimide functionality respectively. Methods of conjugation using engineered cysteines or incorporation of unnatural amino acids have been reported to improve the homogeneity of the conjugate (Axup, J. Y., Bajjuri, K. M., Ritland, M., Hutchins, B. M., Kim, C. H., Kazane, S. A., Halder, R., Forsyth, J. S., Santidrian, A. F., Stafin, K., et al. (2012). Synthesis of site-specific antibody-drug conjugates using unnatural amino acids. Proc. Natl. Acad. Sci. USA 109, 16101-16106; Junutula, J. R., Flagella, K. M., Graham, R. A., Parsons, K. L., Ha, E., Raab, H., Bhakta, S., Nguyen, T., Dugger, D. L., Li, G., et al. (2010). Engineered thio-trastuzumab-DM1 conjugate with an improved therapeutic index to target humanepidermal growth factor receptor 2-positive breast cancer. Clin. Cancer Res. 16, 4769-4778.). Junutula et al. (2008) developed cysteine-based site-specific conjugation called “THIOMABs” (TDCs) that are claimed to display an improved therapeutic index as compared to conventional conjugation methods. Conjugation to unnatural amino acids that have been incorporated into the antibody is also being explored for ADCs; however, the generality of this approach is yet to be established (Axup et al., 2012). In particular the one skilled in the art can also envisage Fc-containing polypeptide engineered with an acyl donor glutamine-containing tag (e.g., Gin-containing peptide tags or Q-tags) or an endogenous glutamine that are made reactive by polypeptide engineering (e.g., via amino acid deletion, insertion, substitution, or mutation on the polypeptide). Then a transglutaminase, can covalently crosslink with an amine donor agent (e.g., a small molecule comprising or attached to a reactive amine) to form a stable and homogenous population of an engineered Fc-containing polypeptide conjugate with the amine donor agent being site-specifically conjugated to the Fc-containing polypeptide through the acyl donor glutamine-containing tag or the accessible/exposed/reactive endogenous glutamine (WO 2012059882). Other methods for conjugating antibodies are also described in WO/2013/092998 and WO2013092983.

Diagnostic Uses:

A further object of the invention relates to an anti-CLDN1 antibody of the invention for diagnosing and/or monitoring disease, and in particular a disease wherein CLDN1 levels are modified (increase or decrease). Typically the disease is cancer and in particular colorectal cancer.

In a preferred embodiment, antibodies of the invention may be labelled with a detectable molecule or substance, such as a fluorescent molecule, a radioactive molecule or any others labels known in the art as above described. For example, an antibody of the invention may be labelled with a radioactive molecule by any method known to the art. For example radioactive molecules include but are not limited radioactive atom for scintigraphic studies such as I123, I124, In111, Re186, Re188. Antibodies of the invention may be also labelled with a spin label for nuclear magnetic resonance (NMR) imaging (also known as magnetic resonance imaging, mri), such as iodine-123, iodine-131, indium-Ill, fluorine-19, carbon-13, nitrogen-15, oxygen-17, gadolinium, manganese or iron. Following administration of the antibody, the distribution of the antibody within the patient is detected. Methods for detecting distribution of any specific label are known to those skilled in the art and any appropriate method can be used. Some non-limiting examples include, computed tomography (CT), position emission tomography (PET), magnetic resonance imaging (MRI), fluorescence, chemiluminescence and sonography.

Antibodies of the invention may be useful for diagnosing and staging of a cancer associated with CLDN1 overexpression (e.g., in radioimaging). Cancer diseases associated with CLDN1 overexpression typically include but are not limited to colorectal cancer, gynaecological cancers (Szabó et al., 2009), ovarian cancers (English and Santin 2013), cervical neoplasias (Sobel, 2005), melanoma (Leotlela et al., 2007), several squamous cell carcinoma (SCC) as oral SCC (Dos Reis et al., 2008), lower lip SCC (de Aquino, 2012), head and neck, skin SCC (Ouban, 2012), Tonsillar SCC (Kondoh, 2011), gastric adenocarcinoma (Wu et al., 2008, Resnick, 2005), thyroid carcinoma (Németh et al., 2010), mammary carcinoma (Myal et al., 2010), Neuroepithelial papillary tumor of the pineal region (PTPR) (Montange 2012), clear cell renal cell carcinoma (Shin et al., 2011), mucoepidermoid carcinoma (MEC) of salivary gland (Aro, 2011), nasopharyngeal carcinoma (Hsueh 2010), urothelial carcinoma of the upper urinary tract (Nakanishi, 2008), esophageal carcinoma (Takala, 2007), mesotheliomas, pleural metastatic adenocarcinoma (Soini, 2006), some pancreas tumors (Tsukahara, 2005), metastatic prostate carcinoma (Szász et al., 2009), lung adenocarcinoma (Chao et al., 2009), breast carcinoma (Lu et al., 2012). It has also been speculated that increased CLDN1 expression may be involved in the early stages of transformation in ulcerative colitis-associated neoplasia (UC) (Kinugasa et al., 2010) and in inflammatory bowel disease-associated neoplasia (Weber CR). CLDN1 protein may therefore be a good candidate for surveillance of these patients.

Antibodies of the invention may be useful for diagnosing diseases other than cancers for which CLDN1 expression is increased or decreased (soluble or cellular CLDN1 form).

Typically, said diagnostic methods involve use of biological sample obtained from the patient. As used herein the term “biological sample” encompasses a variety of sample types obtained from a subject and can be used in a diagnostic or monitoring assay. Biological samples include but are not limited to blood and other liquid samples of biological origin, solid tissue samples such as a biopsy specimen or tissue cultures or cells derived therefrom, and the progeny thereof. For example, biological samples include cells obtained from a tissue sample collected from an individual suspected of having a cancer disease associated with CLDN1 overexpression, and in a preferred embodiment from colorectal cancer. Therefore, biological samples encompass clinical samples, cells in culture, cell supernatants, cell lysates, serum, plasma, biological fluid, and tissue samples.

In a particular embodiment, the invention is a method of diagnosing a cancer disease associated with CLDN1 overexpression in a subject by detecting CLDN1 on cells from the subject using the antibody of the invention. In particular, said method of diagnosing may comprise the steps consisting of (a) contacting a biological sample of a subject likely to suffer from a cancer disease associated with CLDN1 overexpression with an antibody according to the invention in conditions sufficient for the antibody to form complexes with cells of the biological sample that express CLDN1; and (b) detecting and/or quantifying said complexes, whereby the detection of said complexes is indicative of a cancer disease associated with CLDN1 overexpression.

In order to monitor the cancer disease, the method of diagnosing according to the invention may be repeated at different intervals of time, in order to determine if antibody binding to the samples increases or decreases, whereby it is determined if the cancer disease progresses or regresses.

Typically, the antibody may be used in an immunohistochemistry (IHC) method. IHC specifically provides a method of detecting targets in a sample or tissue specimen in situ. The overall cellular integrity of the sample is maintained in IHC, thus allowing detection of both the presence and location of the targets of interest (e.g. CLDN1). Typically a sample is fixed with formalin, embedded in paraffin and cut into sections for staining and subsequent inspection by light microscopy. Current methods of IHC use either direct labeling or secondary antibody-based or hapten-based labeling. Examples of known IHC systems include, for example, EnVision™ (DakoCytomation), Powervision® (Immunovision, Springdale, Ariz.), the NBA™ kit (Zymed Laboratories Inc., South San Francisco, Calif.), HistoFine® (Nichirei Corp, Tokyo, Japan). In particular embodiment, a tissue section (e.g. a sample comprising cumulus cells) may be mounted on a slide or other support after incubation with the anti-CLDN1 antibody. Then, microscopic inspections in the sample mounted on a suitable solid support may be performed. For the production of photomicrographs, sections comprising samples may be mounted on a glass slide or other planar support, to highlight by selective staining CLDN1. Therefore IHC samples may include, for instance: (a) preparations comprising cumulus cells (b) fixed and embedded said cells and (c) detecting CLDN1 in said cells samples. In some embodiments, an IHC staining procedure may comprise steps such as: cutting and trimming tissue, fixation, dehydration, paraffin infiltration, cutting in thin sections, mounting onto glass slides, baking, deparaffination, rehydration, antigen retrieval, blocking steps, applying primary antibodies (i.e. anti-CLDN1 antibodies), washing, applying secondary antibodies (optionally coupled to a suitable detectable label), washing, counter staining, and microscopic examination.

Therapeutic Uses:

Antibodies, fragments or immunoconjugates of the invention may be useful for treating any disease associated with CLDN1 expression. The antibodies of the invention may be used alone or in combination with any suitable agent.

In one embodiment, the anti-CLDN1 antibody of the invention (or the immunoconjugate comprising thereof) may be used for the treatment of cancer, in particular colorectal cancer. Cancer diseases associated with CLDN1 overexpression typically include but are not limited to colorectal cancer, gynaecological cancers (Szabó et al., 2009), ovarian cancers (English and Santin 2013), cervical neoplasias (Sobel, 2005), melanoma (Leotlela et al., 2007), squamous cell carcinoma (SCC) as oral SCC(Dos Reis et al., 2008), lower lip SCC (de Aquino, 2012), head and neck, skin SCC (Ouban, 2012), Tonsillar SCC (Kondoh, 2011), gastric adenocarcinoma (Wu et al., 2008, Resnick, 2005), thyroid carcinoma (Németh et al., 2010), mammary carcinoma (Myal et al., 2010), Neuroepithelial papillary tumor of the pineal region (PTPR) (Montange 2012), clear cell renal cell carcinoma (Shin et al., 2011), mucoepidermoid carcinoma (MEC) of salivary gland (Aro, 2011), nasopharyngeal carcinoma (Hsueh 2010), urothelial carcinoma of the upper urinary tract (Nakanishi, 2008), esophageal carcinoma (Takala, 2007), mesotheliomas, pleural metastatic adenocarcinoma (Soini, 2006), some pancreas tumors (Tsukahara, 2005).

It is well known that therapeutic monoclonal antibodies can lead to the depletion of cells bearing the antigen specifically recognized by the antibody. This depletion can be mediated through at least three mechanisms: antibody mediated cellular cytotoxicity (ADCC), complement dependent lysis, and direct anti-tumour inhibition of tumour growth through signals given via the antigen targeted by the antibody. “Complement dependent cytotoxicity” or “CDC” refers to the lysis of a target cell in the presence of complement. Activation of the classical complement pathway is initiated by the binding of the first component of the complement system to antibodies which are bound to their cognate antigen. To assess complement activation, a CDC assay, e.g. as described in Gazzano-Santoro et al. (1997) may be performed. “Antibody-dependent cell-mediated cytotoxicity” or “ADCC” refers to a form of cytotoxicity in which secreted antibodies bound onto Fc receptors (FcRs) present on certain cytotoxic cells (e.g. Natural Killer (NK) cells, neutrophils, and macrophages) enable these cytotoxic effector cells to bind specifically to an antigen-bearing target cell and subsequently kill the target cell. To assess ADCC activity of a molecule of interest, an in vitro ADCC assay, such as that described in U.S. Pat. Nos. 5,500,362 or 5,821,337 may be performed.

Anti-Claudin-1 antibodies of the present invention may also be used in therapeutic and prophylactic methods to treat and/or prevent HCV infection. The anti-Claudin-1 antibody interferes with HCV-host cells interactions by binding to the extracellular domain of Claudin-1 on a cell surface, thereby reducing, inhibiting, blocking or preventing HCV entry into the cell and/or HCV infection of the cell (WO2010034812). Antibodies of the present invention may be used in a variety of therapeutic or prophylactic methods. In particular, the present invention provides a method for treating or preventing a liver disease or pathology in a subject, which comprises administering to the subject an effective amount of an antibody of the invention which inhibits HCV from entering or infecting the subject's cells, so as to thereby treat or prevent the liver disease or pathology in the subject. The liver disease or pathology may be inflammation of the liver, liver fibrosis, cirrhosis, and/or hepatocellular carcinoma (i.e., liver cancer) associated with HCV infection. The present invention also provides a method for treating or preventing a HCV-associated disease or condition (including a liver disease) in a subject, which comprises administering to the subject an effective amount of an antibody of the invention which inhibits HCV from entering or infecting the subject's cells, so as to thereby treat or prevent the HCV-associated disease or condition in the subject. In certain embodiments of the present invention, the antibody or composition is administered to a subject diagnosed with acute hepatitis C. In other embodiments of the invention, the antibody or composition is administered to a subject diagnosed with chronic hepatitis C. In one embodiment, the methods of the present invention may be used to reduce the likelihood of a subject's susceptible cells of becoming infected with HCV as a result of liver transplant. As already mentioned above, when a diseased liver is removed from a HCV-infected patient, serum viral levels plummet. However, after receiving a healthy liver transplant, virus levels rebound and can surpass pre-transplant levels within a few days. Liver transplant patients may benefit from administration of an inventive antibody that binds to the ectodomain of Claudin-1 on the surface of hepatocytes and thereby reduce, inhibit, block or prevent HCV entry into the cells. Administration may be performed prior to liver transplant, during liver transplant, and/or following liver transplant.

In each of the embodiments of the treatment methods described herein, the anti-CLDN1 monoclonal antibody or anti-CLDN1 monoclonal antibody-drug conjugate is delivered in a manner consistent with conventional methodologies associated with management of the disease or disorder for which treatment is sought. In accordance with the disclosure herein, an effective amount of the antibody or antibody-drug conjugate is administered to a subject in need of such treatment for a time and under conditions sufficient to prevent or treat the disease or disorder.

Thus, an object of the invention relates to a method for treating a disease associated with the expression of CLDN1 comprising administering a subject in need thereof with a therapeutically effective amount of an antibody, fragment or immunoconjugate of the invention.

In the context of the invention, the term “treating” or “treatment”, as used herein, means reversing, alleviating, inhibiting the progress of, or preventing the disorder or condition to which such term applies, or one or more symptoms of such disorder or condition.

According to the invention, the term “patient” or “patient in need thereof” is intended for a human affected or likely to be affected with disease associated with overexpression of CLDN1.

By a “therapeutically effective amount” of the antibody of the invention is meant a sufficient amount of the antibody to treat said cancer, at a reasonable benefit/risk ratio applicable to any medical treatment. It will be understood, however, that the total daily usage of the antibodies and compositions of the present invention will be decided by the attending physician within the scope of sound medical judgment. The specific therapeutically effective dose level for any particular patient will depend upon a variety of factors including the disorder being treated and the severity of the disorder; activity of the specific antibody employed; the specific composition employed, the age, body weight, general health, sex and diet of the patient; the time of administration, route of administration, and rate of excretion of the specific antibody employed; the duration of the treatment; drugs used in combination or coincidental with the specific antibody employed; and like factors well known in the medical arts. For example, it is well known within the skill of the art to start doses of the compound at levels lower than those required to achieve the desired therapeutic effect and to gradually increase the dosage until the desired effect is achieved.

In certain embodiments, an anti-CLDN1 monoclonal antibody or antibody-drug conjugate is used in combination with a second agent for treatment of a disease or disorder. When used for treating cancer, an anti-CLDN1 monoclonal antibody or antibody-drug conjugate of the present invention may be used in combination with conventional cancer therapies such as, e.g., surgery, radiotherapy, chemotherapy, or combinations thereof. In certain aspects, other therapeutic agents useful for combination cancer therapy with an anti-CLDN1 antibody or antibody-drug conjugate in accordance with the present invention include anti-angiogenic agents. In some aspects, an antibody or antibody-drug conjugate in accordance with the present invention is co-administered with a cytokine (e.g., a cytokine that stimulates an immune response against a tumor). In some embodiments, an anti-CLDN1 monoclonal antibody or antibody-drug conjugate as described herein is used in combination with a tyrosine kinase inhibitor (TKI). In some embodiments, an anti-CLDN1 monoclonal antibody or antibody-drug conjugate as described herein is used in combination with another therapeutic monoclonal antibody (mAb). Trastuzumab (Herceptin, Roche), Bevacizumab (Avastin, Roche) and Cetuximab (Erbitux, Merck) are three such mAb that have been approved. Other mAb include, but are not limited to: Infliximab (Remicade, Johnson&Johnson), Rituximab (Rituxan, Roche), Adalimumab (Humira, Abbott) and Natalizumab (Tysabri, Biogen).

Pharmaceutical Compositions:

For administration, the anti-CLDN1 monoclonal antibody or antibody-drug conjugate is formulated as a pharmaceutical composition. A pharmaceutical composition comprising an anti-CLDN1 monoclonal antibody or antibody-drug conjugate can be formulated according to known methods to prepare pharmaceutically useful compositions, whereby the therapeutic molecule is combined in a mixture with a pharmaceutically acceptable carrier. A composition is said to be a “pharmaceutically acceptable carrier” if its administration can be tolerated by a recipient patient. Sterile phosphate-buffered saline is one example of a pharmaceutically acceptable carrier. Other suitable carriers are well-known to those in the art. (See, e.g., Gennaro (ed.), Remington's Pharmaceutical Sciences (Mack Publishing Company, 19th ed. 1995)) Formulations may further include one or more excipients, preservatives, solubilizers, buffering agents, albumin to prevent protein loss on vial surfaces, etc.

The form of the pharmaceutical compositions, the route of administration, the dosage and the regimen naturally depend upon the condition to be treated, the severity of the illness, the age, weight, and sex of the patient, etc.

The pharmaceutical compositions of the invention can be formulated for a topical, oral, parenteral, intranasal, intravenous, intramuscular, subcutaneous or intraocular administration and the like.

Preferably, the pharmaceutical compositions contain vehicles which are pharmaceutically acceptable for a formulation capable of being injected. These may be in particular isotonic, sterile, saline solutions (monosodium or disodium phosphate, sodium, potassium, calcium or magnesium chloride and the like or mixtures of such salts), or dry, especially freeze-dried compositions which upon addition, depending on the case, of sterilized water or physiological saline, permit the constitution of injectable solutions.

The doses used for the administration can be adapted as a function of various parameters, and in particular as a function of the mode of administration used, of the relevant pathology, or alternatively of the desired duration of treatment.

To prepare pharmaceutical compositions, an effective amount of the antibody may be dissolved or dispersed in a pharmaceutically acceptable carrier or aqueous medium.

The pharmaceutical forms suitable for injectable use include sterile aqueous solutions or dispersions; formulations including sesame oil, peanut oil or aqueous propylene glycol; and sterile powders for the extemporaneous preparation of sterile injectable solutions or dispersions. In all cases, the form must be sterile and must be fluid to the extent that easy syringability exists. It must be stable under the conditions of manufacture and storage and must be preserved against the contaminating action of microorganisms, such as bacteria and fungi.

Solutions of the active compounds as free base or pharmacologically acceptable salts can be prepared in water suitably mixed with a surfactant, such as hydroxypropylcellulose. Dispersions can also be prepared in glycerol, liquid polyethylene glycols, and mixtures thereof and in oils. Under ordinary conditions of storage and use, these preparations contain a preservative to prevent the growth of microorganisms.

An antibody of the invention can be formulated into a composition in a neutral or salt form. Pharmaceutically acceptable salts include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like.

The carrier can also be a solvent or dispersion medium containing, for example, water, ethanol, polyol (for example, glycerol, propylene glycol, and liquid polyethylene glycol, and the like), suitable mixtures thereof, and vegetables oils. The proper fluidity can be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminium monostearate and gelatin.

Sterile injectable solutions are prepared by incorporating the active compounds in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

The preparation of more, or highly concentrated solutions for direct injection is also contemplated, where the use of DMSO as solvent is envisioned to result in extremely rapid penetration, delivering high concentrations of the active agents to a small tumor area.

Upon formulation, solutions will be administered in a manner compatible with the dosage formulation and in such amount as is therapeutically effective. The formulations are easily administered in a variety of dosage forms, such as the type of injectable solutions described above, but drug release capsules and the like can also be employed.

For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous and intraperitoneal administration. In this connection, sterile aqueous media which can be employed will be known to those of skill in the art in light of the present disclosure. For example, one dosage could be dissolved in 1 ml of isotonic NaCl solution and either added to 1000 ml of hypodermoclysis fluid or injected at the proposed site of infusion, (see for example, “Remington's Pharmaceutical Sciences” 15th Edition, pages 1035-1038 and 1570-1580). Some variation in dosage will necessarily occur depending on the condition of the subject being treated. The person responsible for administration will, in any event, determine the appropriate dose for the individual subject.

The antibodies of the invention may be formulated within a therapeutic mixture to comprise about 0.0001 to 1.0 milligrams, or about 0.001 to 0.1 milligrams, or about 0.1 to 1.0 or even about 10 milligrams per dose or so. Multiple doses can also be administered.

In addition to the compounds formulated for parenteral administration, such as intravenous or intramuscular injection, other pharmaceutically acceptable forms include, e.g. tablets or other solids for oral administration; time release capsules; and any other form currently used.

In certain embodiments, the use of liposomes and/or nanoparticles is contemplated for the introduction of antibodies into host cells. The formation and use of liposomes and/or nanoparticles are known to those of skill in the art.

Nanocapsules can generally entrap compounds in a stable and reproducible way. To avoid side effects due to intracellular polymeric overloading, such ultrafine particles (sized around 0.1 μm) are generally designed using polymers able to be degraded in vivo. Biodegradable polyalkyl-cyanoacrylate nanoparticles that meet these requirements are contemplated for use in the present invention, and such particles may be are easily made.

Liposomes are formed from phospho lipids that are dispersed in an aqueous medium and spontaneously form multilamellar concentric bilayer vesicles (also termed multilamellar vesicles (MLVs)). MLVs generally have diameters of from 25 nm to 4 μm. Sonication of MLVs results in the formation of small unilamellar vesicles (SUVs) with diameters in the range of 200 to 500 Å, containing an aqueous solution in the core. The physical characteristics of liposomes depend on pH, ionic strength and the presence of divalent cations.

Kits:

Finally, the invention also provides kits comprising at least one antibody of the invention. Kits containing antibodies of the invention find use in detecting CLDN1 expression (increase or decrease), or in therapeutic or diagnostic assays. Kits of the invention can contain an antibody coupled to a solid support, e.g., a tissue culture plate or beads (e.g., sepharose beads). Kits can be provided which contain antibodies for detection and quantification of CLDN1 in vitro, e.g. in an ELISA, Western blot or IHC. Such antibody useful for detection may be provided with a label such as a fluorescent or radiolabel.

The invention will be further illustrated by the following figures and examples. However, these examples and figures should not be interpreted in any way as limiting the scope of the present invention.

Figures:

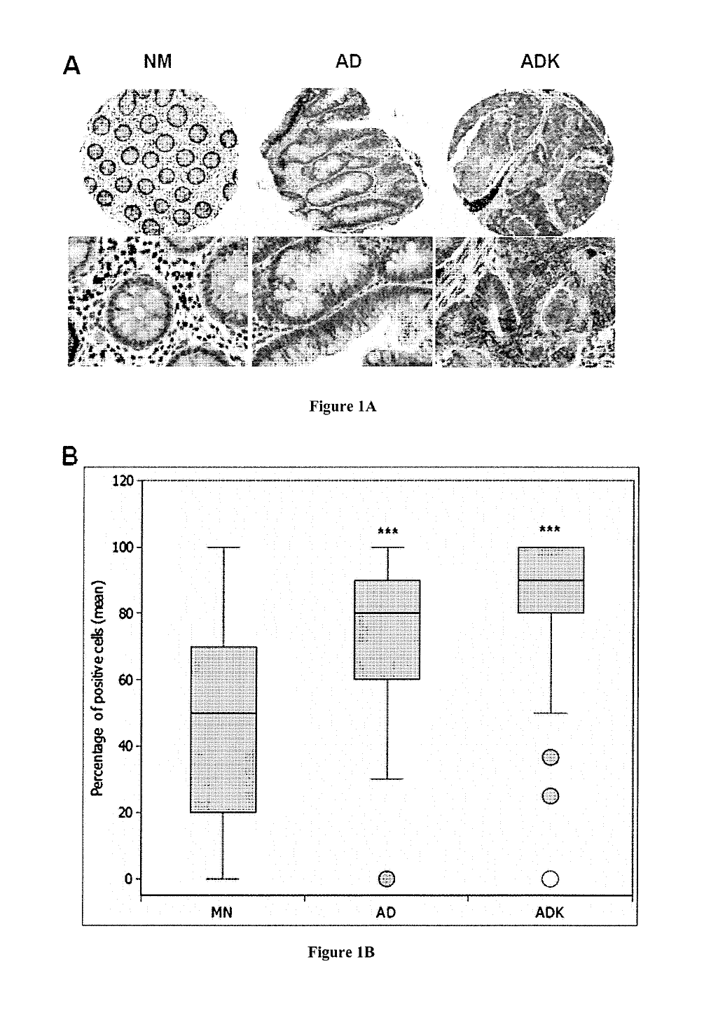

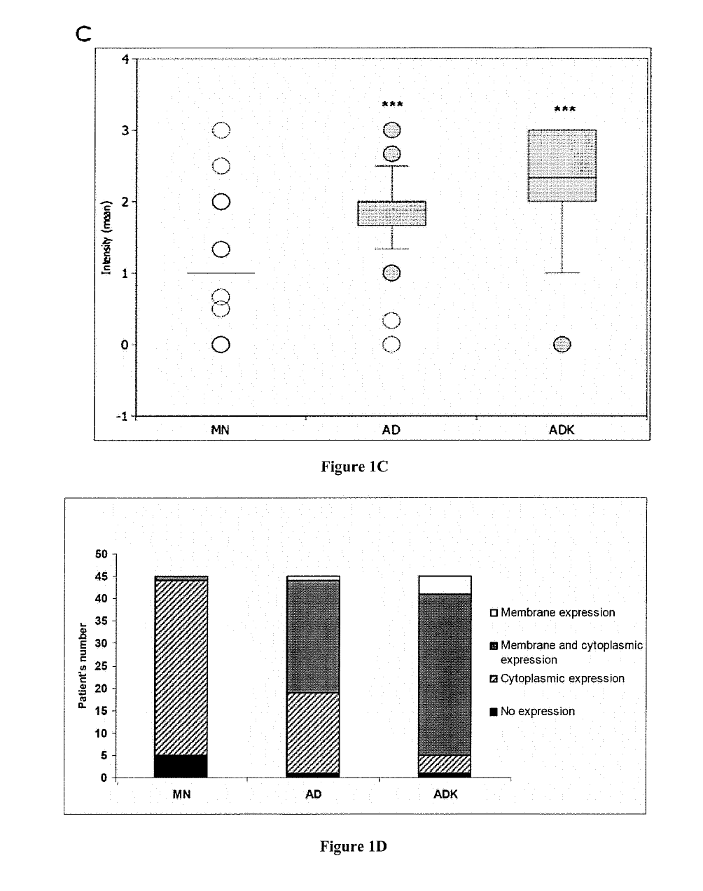

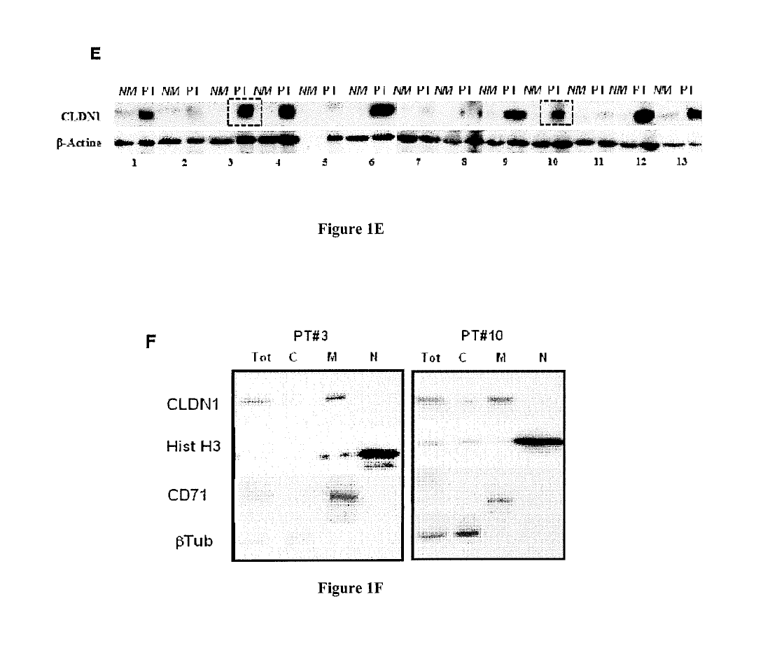

FIG. 1 shows CLDN1 immunochemistry on colon clinical samples A) Example of CLDN1 staining in the three types of colon tissue NM=normal mucosa; AD=Adenoma; ADK=adenocarcinoma (X100) B) CLDN1 expression assessed as labeling intensity or C) as % of labeled cells. D) Localisation of the CLDN1 is indicated for each tissue of the 45 colorectal patients. E) Western blot analysis of CLDN1 expression from 13 matched tissue samples. NM=normal mucosa; PT=primary tumor. F) Subcellular fractionation of two primary tumor sample. C=cytoplasm, M=membrane, N=nucleus. β-tubulin, CD71 and Histone H3 were used as subcellular markers.

FIG. 2 shows the reactivity against CLDN1 of the three hybridomas selected. FACS histograms showing binding of selected hybridomas to CLDN1-positive cell lines (SW480-CLDN1 and SW620shLUC).

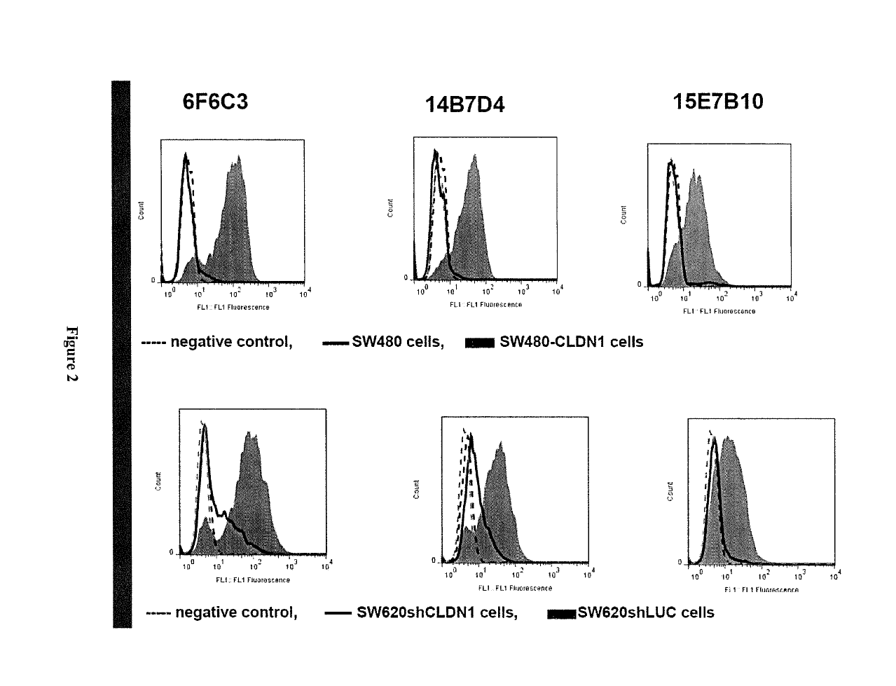

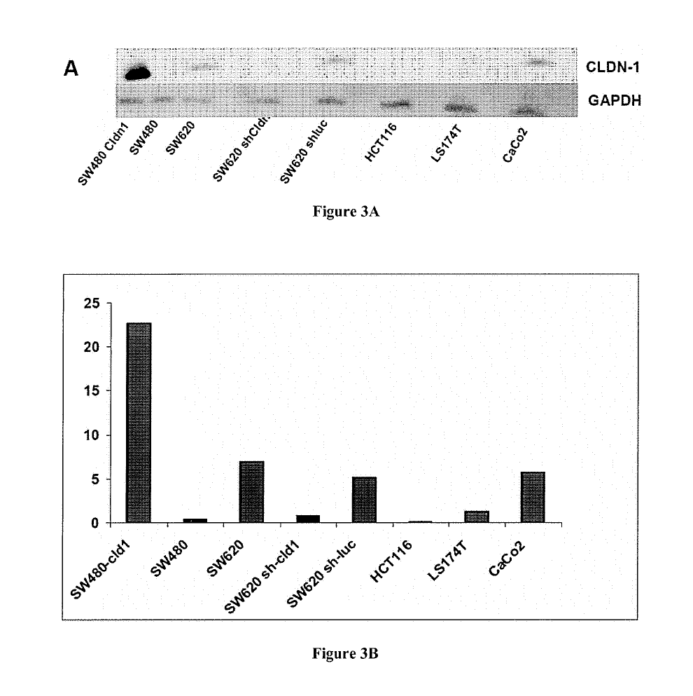

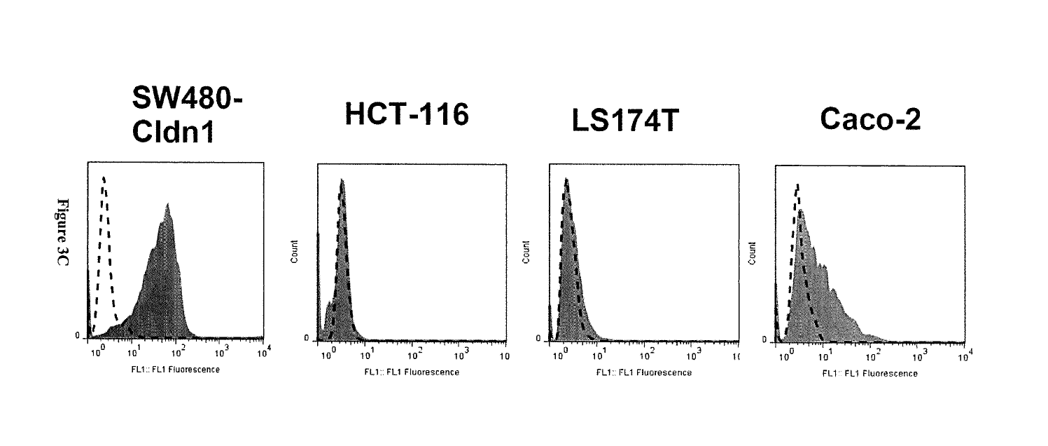

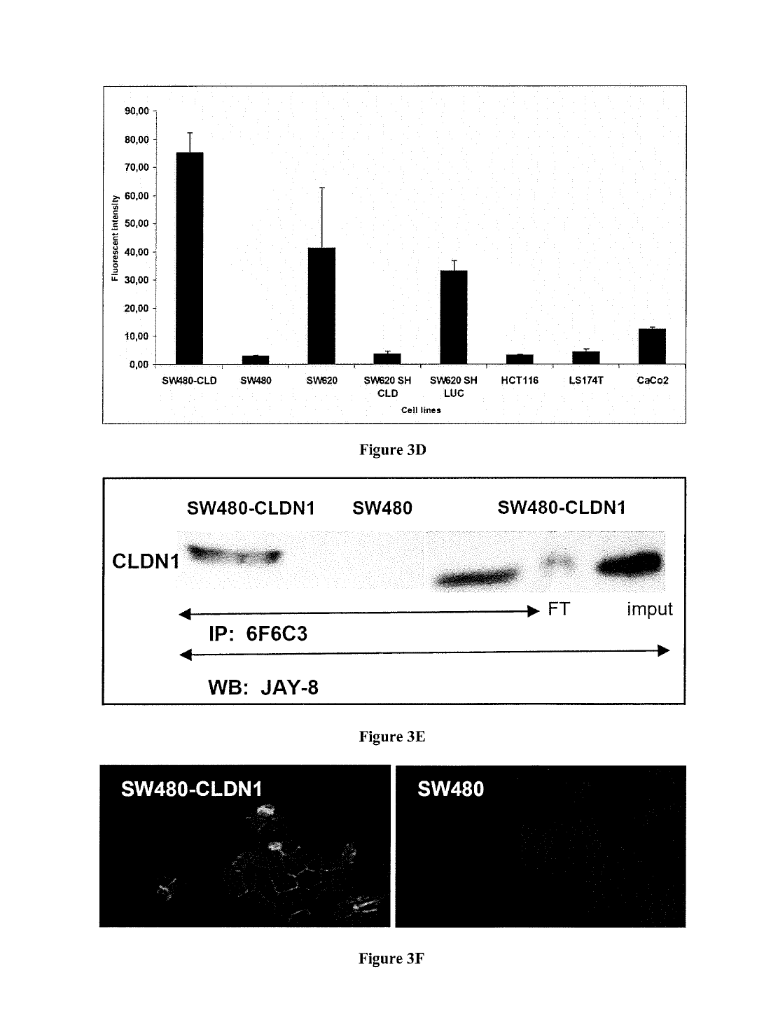

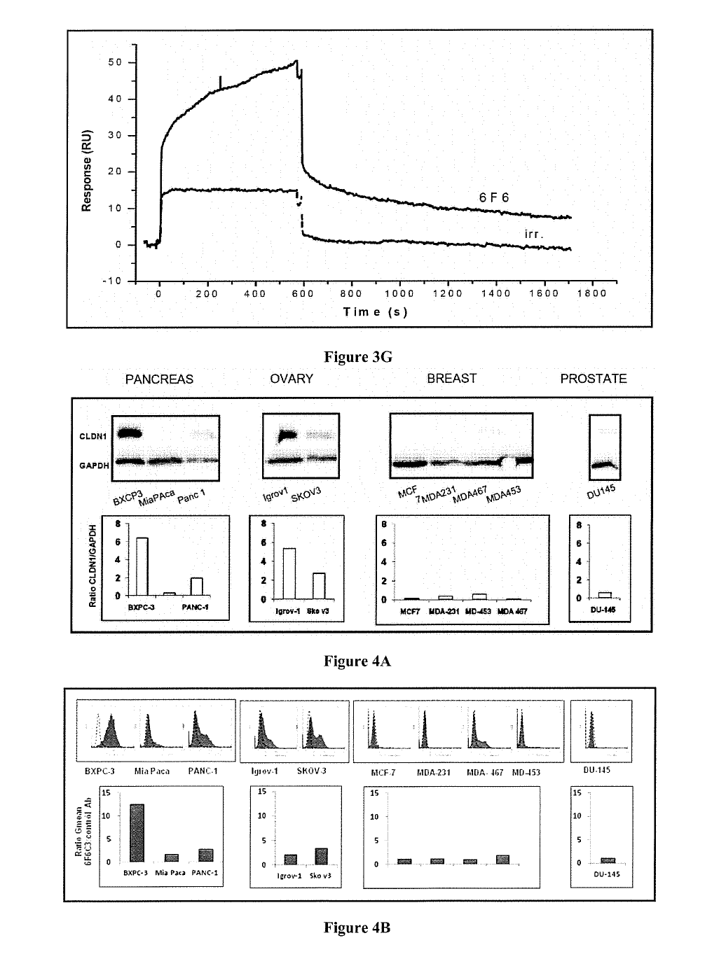

FIG. 3 shows reactivity and specificity of 6F6C3 mAb against CLDN1. A) CLDN1 expression evaluated by western blotting in different colorectal cell lines. B) GAPDH-normalized expression of CLDN1 using GeneSnap fom Syngene. C) Reactivity of purified 6F6C3 mAb (10 μg/ml) on the negative and positive-CLDN1 cell lines, determined by FACs experiments. D) The fluorescence intensities of 6F6C3 mAb binding are presented as the mean±SD of at least 3 independent experiments. E) Immunoprecipitation of CLDN1 from SW480 and SW480-CLDN1 with 6F6C3 mAb; the complex was revealed by JAY-8 anti-CLDN1 antibody (IP=immunoprecipitation, FT=flow through). F) Immunofluorescence experiments were performed using 6F6C3 mAb as primary antibody. Images were recorded using a 63XNA objective on a Leica inverted microscope. G) Surface plasmon resonance measurements of the interaction of 6F6C3 and irrelevant antibodies on CLDN1-membrane extracts. The binding of 6F6C3 and irrelevant antibodies were performed on a Biacore 3000 instrument at 25° C. in PBS. Membrane extracts were immobilized at 2600 RU on HPA sensor chip surface according to the manufacturer's specifications. Irrelevant and 6F6C3 antibodies were injected at 660 nM over the immobilized LPS at a flow rate of 2 μL/min during 10 min followed by a 600 s dissociation step with PBS running buffer.

FIG. 4 shows the CLDN1 expression of several cancer cell lines and the reactivity of 6F6C3 mAb. A) Total CLDN1 expression determined by Western blot using polyclonal anti-CLDN1 antibody (JAY-8) Histograms represent the ratio CLDN1/GAPDH to normalize expression. B) Reactivity of 6F6C3 mAb (gray) or irrelevant Ab (dotted line) against cell lines. Quantification was done using Gmean ratio of 6F6C3 mAb and irrelevant antibody.

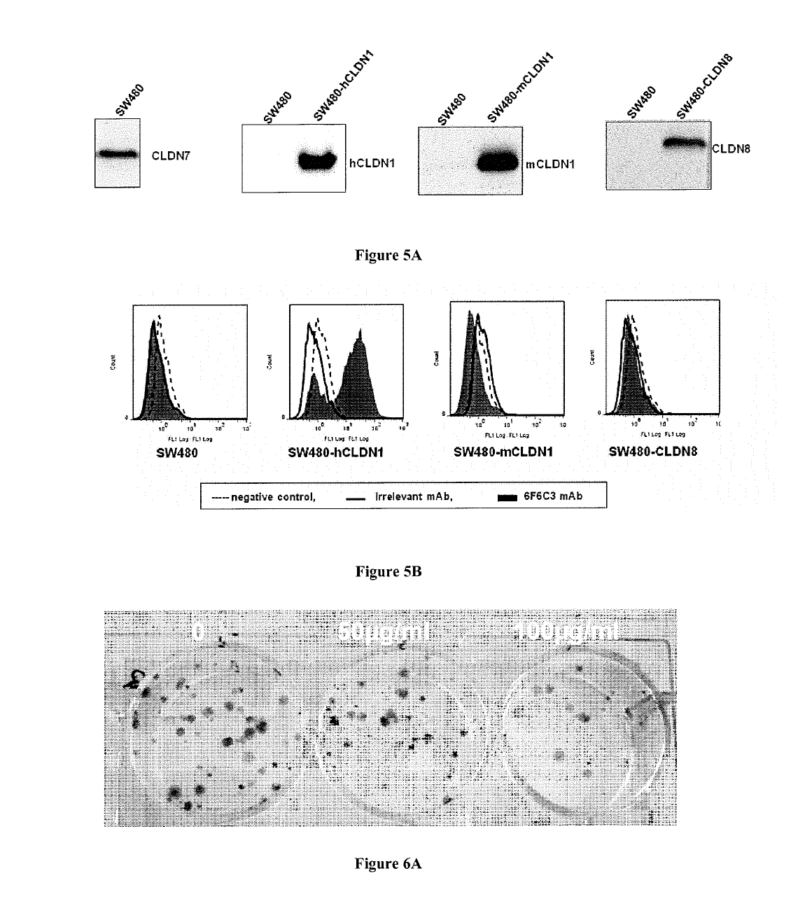

FIG. 5 shows cross-reactivity analysis of 6F6C3 mAb against other CLDNs. A). Cell lysates derived from SW480 or CLDN-transfected SW480 were tested by Western-Blotting. B) FACS histograms of the binding 6F6C3 mAb (10 μg/mL-gray histogram) or control without 6F6C3 (dashed histogram) or irrelevant antibody 35A7 (black histogram) to the different CLDNs.

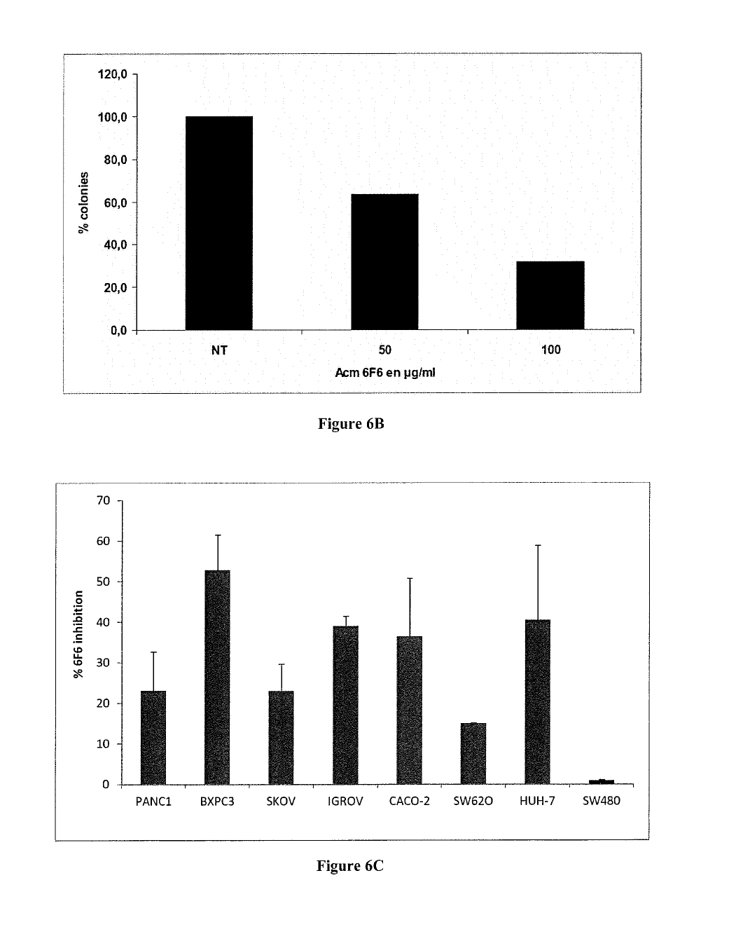

FIG. 6 shows the in vitro effect of 6F6C3 mAb on cell lines survival. A) clonogenic assay on Caco-2 colorectal cell line: 250 cells are seeded on a 6-well plate and allowed to adhere overnight at 37° C. Then, one milliliter of RPMI with or without antibody (final concentration of 50 or 100 μg/ml) was added and incubated for 6 days. After 6 days more in free-medium, plates were washed; colonies were fixed (ethanol/acetic acid), stained with crystal violet (0.5% w/v) and counted using a stereomicroscope. B) Percentage of colonies of Caco2 cell line with and without treatment C) Clonogenic assay on several cell lines treated or not with 6F6C3 mAb at 100 μg/ml; histograms represent the % of inhibition (% colonies in no treated well—% colonies in treated well).

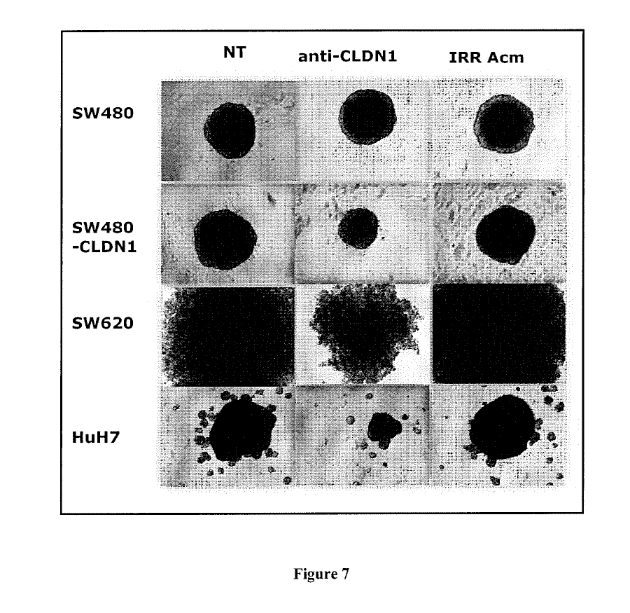

FIG. 7 shows effect of 6F6C3 mAb on 3D cell line growth. The treated cells were incubated 2 h with the mAb at 50 μg/ml (6F6C3mAb or irrelevant mAb) before seeding. Representative images of spheroids grown on Ultra low attachment plates were taken after 96 h of growth.

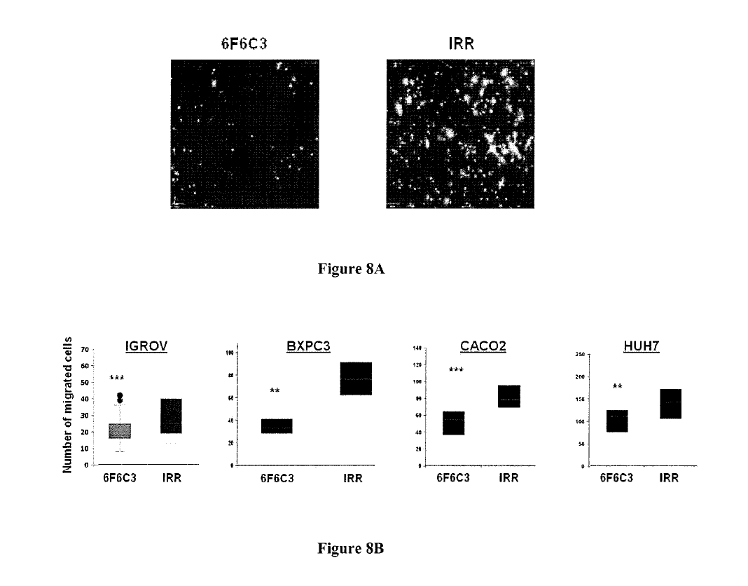

FIG. 8 shows migration assay using Boyden chambers. A) Photographs of HUH-7 cells treated or not by 6F6C3mAb at 100 μg/ml cells from the underside of Boyden chamber membrane. B) Number of migrated cells in three independent experiments for the 4 cell lines treated with 100 μg/ml of 6F6C3mAb or irrelevant mAb. Cells were preincubated with mAb 1 hour before loading.

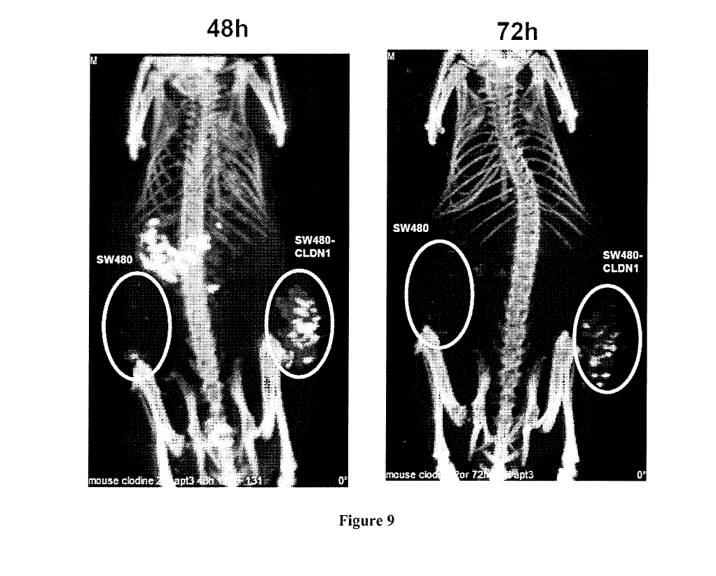

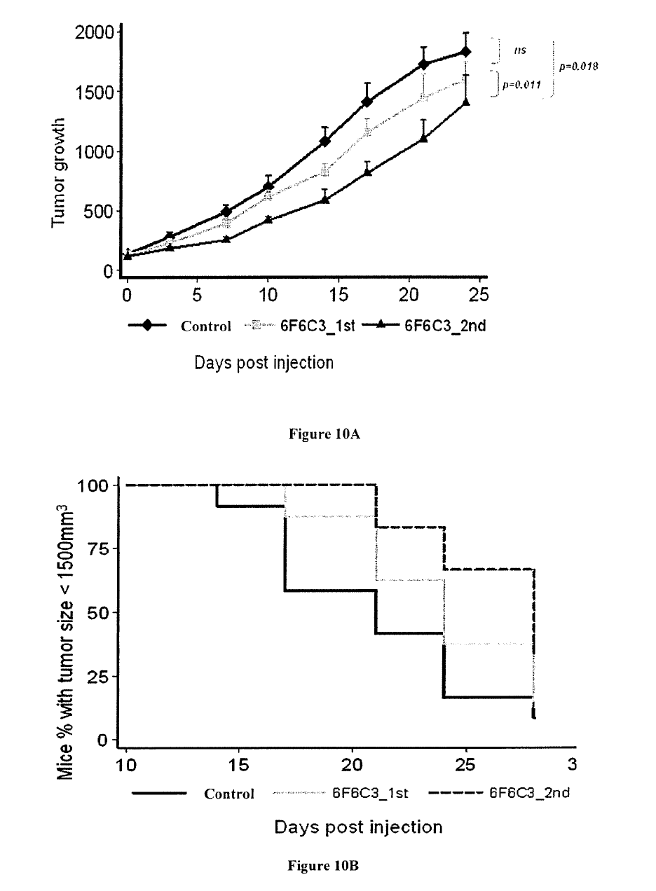

FIG. 9 shows biodistribution of ¹²⁵I 6F6C3mAb. Grafted-mice were given intravenous injections via tail vein of 500 μCi of ¹²⁵I 6F6C3mAb and images were acquired 2 days and 3 days after injection.

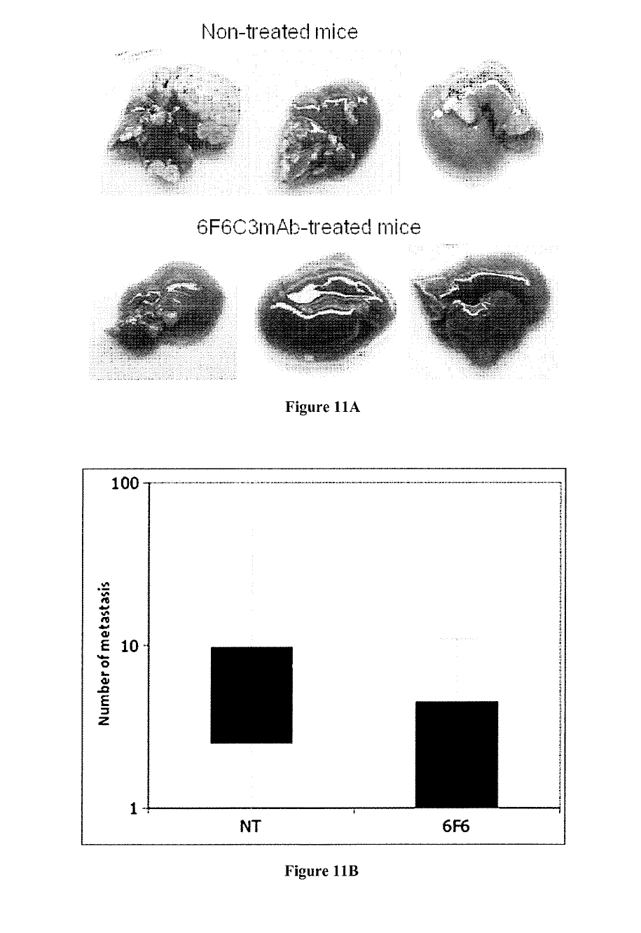

FIG. 10 shows a study of the in vivo effect of 6F6C3 mAb on the growth of SW620 xenografts in athymic nude mice. A) Tumor growth kinetics of xenografted mice with SW620 treated or not (black line) by 6F6C3 mAb at 15 mg/kg twice a week (gray line) or at 15 mg/kg three times a week (dark gray line). Treatment started when tumors reach 100 mm³. B) An adapted Kaplan-Meier curves using the time taken for the tumour to reach a determined volume of 1500 mm³. Black solid line corresponds to NT (non treated), gray solid line corresponds to the first experiment and gray dotted line to the second′ one.

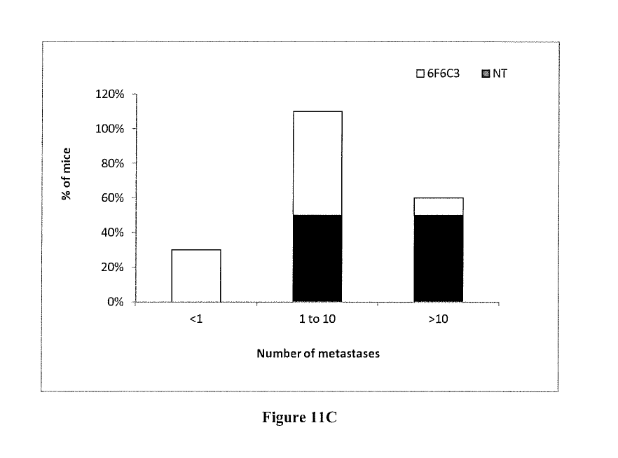

FIG. 11 shows the in vivo effect of 6F6C3 mAb on the formation of liver metastases. A) Representative SW620 metastatic tumors in liver from non-treated and 6F6C3 mAb treated mice; images were taken at the experimental endpoint (5 weeks from surgery). B) Comparison of the distribution and median of the number of metastases between both groups (p=0.08, Mann-Whitney). C) Repartition of the mice according to the number of liver metastases. <1=no metastasis or one micro metastasis; 1-10=more than 1 and less than 10 metastases; >10=more than 10 metastases.

### EXAMPLE

### Material & Methods

1—CLDN1 Immunochemistry on Colon Clinical Samples

Tissue micro-array (TMA) was constructed as previously described (Granci et al., 2008), using 3 tissue cores (0.6-mm diameter each) of colon cancer, of matched normal mucosa and of matched adenoma from 52 patients. Three-μm thin microns sections of the TMA were de-paraffinized and rehydrated in graded alcohols. The slides were subsequently subjected to heat-induced epitote retrieval by immersing them in a water bath with an EDTA buffer (pH 9). After neutralization of endogenous peroxidase activity, TMA sections were incubated for 60 min. with the polyclonal anti-CLDN1 antibody (JAY-8, Zymed laboratories Inc, CA, USA) or diluent only. Primary antibodies binding was visualized using the Envision® system with the Dako Autostainer® (Dako, Glostrup, Denmark). No staining was observed on the slide incubated with antibody diluent. Among the 52 cases sampled 45 samples with matched normal tissue, adenomas and tumors remains assessable after immunohistochemistry. Each spot was assigned individually for percentage of marked cells and for staining intensity (0: none; 1: faint; 2: moderate; 3 strong).

2—Cell Lines

The human colorectal cancer cell lines used were: SW480 (ATCC CCL-228), SW620 (ATCC CCL-227), Caco-2 (ATCC HTB-37), Difi ((Olive et al., 1993) a gift from Dr Montagut, HCT116 (CCL-247), LS174T (ATCC CL-188).

The other cancer cell lines used were: pancreatic cancer PANC1 (ATCC CRL1469) BXPC3 (ATCC CRL-1687), ovarian cancer SKOV-3 (ATCC HTB-77) IGROV1 (Bénard et., al 1985) and hepatocarcinoma HuH-7 (JCRB0403).

To obtain the CLDN1-positive SW480 cell line (SW480-CLDN1), we stably transfected the SW480 cell line with the human CLDN1 cDNA clone (Invitrogen MGC collection, ref 4500534, pCMV-SPORT6) using jetPRIME™ transfection reagent (Polyplus-transfection Inc., France). The stable clones were generated using geneticin as selection reagent. SW620 cell line expressing ShRNAs targeting luciferase (SW620shLUC), or CLDN1 (SW620shCLDN1) were obtained by retroviral gene transduction of the pSIREN vector. Targeting sequence are: ShLuc (from RNAi-Ready pSIREN-RetroQ vector kit, Clontech Mountain View, Calif., USA). After 24 hours from transduction, cells were selected with 1 μg/mL of puromycin and stable clones were pooled.

All the cell lines were grown in complete medium i.e., RPMI 1640 medium supplemented with 10% heat-inactivated fetal calf serum (FCS) and 2 mM L-glutamine at 37° C. under a humidified atmosphere with 5% CO2, and passaged by trypsinization using trypsin (0.5 mg/mL) EDTA (0.2 mg/mL). All culture medium supplements were purchased from Life Technologies, Inc. (Gibco BRL, Gaithersburg, Md.). For the transfected cells, geneticine (0.67%) was added in the medium.

3—Monoclonal Antibodies:

Anti-CLDN1 mAbs:

Mice hybridomas were generated by immunizing BALB/c mice five times i.p. at 2-week intervals with 4 millions of murine NIH cells transiently transfected with CLDN1 referred as NIH-CLDN1 in complete Freund's adjuvant (Sigma) for the first injection, and incomplete Freund's adjuvant (Sigma) for subsequent injections. An i.v. booster injection of NIH-CLDN1 was given three months after the filth immunization. Three days later, spleen cells from immunized mice were fused with the mouse myeloma cell line P3-X63-Ag.8.653. Supernatants from newly generated clones were screened by fluorescence-activated cell sorting (FACs) using SW480-CLDN1. The specificity for CLDN1 of supernatants was confirmed on CLDN1 positive cells as SW620 colorectal cell line.

MAbs Used as Controls:

In control experiments, anti-CEA monoclonal antibody 35A7 (specific for the CEA Gold 2 epitope, (Haskell et al., 1983; Hammarstrom et al., 1989) and an irrelevant normal mouse IgG3 (sc-3880, Santa Cruz Biotechnology)

4—Western Blot Analysis

Patients tissues samples were directly disrupted in a lysis buffer (NaCl 150 mM, 10 mM Tris, pH 7.4, 1 mM, EDTA, 1 mM EGTA, 1% SDS, 1% Triton X-100, 0.5% NP-40, 2 mM PMSF, 100 mM NaF, 10 mM sodium ortho-vanadate, one cocktail protease inhibitor tablet for 10 ml) using Mixer Mill® MM 300 (Qiagen, Valencia, Calif.). The protein concentration was determined with a Bradford assay (Pierce Coomassie Plus Protein Assay). Then, 50 μg of total protein was resolved by 12% SDS-PAGE and transferred onto nitrocellulose membranes (Whatman® Protran®, pore size 0.45 μm). The nonspecific binding sites were blocked with 5% (wt/vol) nonfat milk in PBS-T (PBS with 0.1% (vol/vol) Tween 20) for 1 hour at room temperature and then incubated overnight at 4° C. with polyclonal anti-CLDN1 antibody (JAY-8). Membranes were then washed and incubated with appropriate horeradish peroxidase-conjugated secondary antibody for 1 hr. Revelation was performed with a Chemiluminescence system (Amersham Biosciences). β-tubuline expression was used to normalisation.

5—Subcellular Protein Extraction from Tissue Samples

For each sample 20-μm thickness slides were cut with a cryotome, mixed, recovered in liquid nitrogen and gently ground with a micropestle. For subcellular protein extraction, the ProteoExtract Subcellular Proteome Extraction Kit was used according to the manufacturer's instructions (Calbiochem). 10 μg of each subcellular fraction were loaded on 12% SDS-PAGE gel. Immunoblotting was done as described above. The following primary antibodies were used: anti-CLDN1 (JAY-8), anti-CD71 (Invitrogen), anti-Histone H3 (Pierce) and anti β-tubuline (Sigma T4026)

6—Flow Cytometry Experiments

Hybridomas or mAbs binding was determined with a FACScan fluorescence-activated cell sorter (Quanta apparatus, Beckman Coulter). Cells were seeded in 25 cm² flasks (2×10⁵ cells/flask). After a 48-hour rest, one million cells were pelleted, Washed with PBS-1% BSA and incubated with hybridomas or mAbs, on ice for 1 h. After washing, an appropriate anti-mouse FITC conjugated monoclonal antibody ((1:60 dilution; (Invitrogen) was added (on ice for 45 mn) to detect the primary antibodies. Direct incubation of cells with the secondary antibody was used for background measurements (negative control).

7—Immunoprecipitation Studies

SW480-CLDN1 and SW480 cell culture dishes were washed with cold PBS, then adherent cells were scrapped using cold lysis buffer (25 mM Tris-HCl, 150 mM NaCl, 1 mM EDTA, 1% NP40 and one tablet of protease's inhibitors). After centrifugation, 200 μg of cell lysate was mixed with 1 ml of 6F6C3 hybridoma culture supernactant and 100 μl of G sepharose beads. The lysate beads mixture was incubated for 2 h at 4° C. under rotary agitation. The complex was eluted from beads and runned on a gel for western Blot. CLDN1 revelation was done using a commercial anti-CLDN1 antibody (JAY-8).

8—Immunofluorescence Studies

The cells were plated in culture dishes containing 12 mm glass coverslips. One day after plating, cells on the coverslip were fixed with 4% paraformaldhehyde/PBS at room temperature for 10 min and blocked with PBS containing 5% BSA at 37° C. for 30 min. The cells were incubated with 6F6C3 mAb (10 μg/ml) for 1 h. Secondary antibody was a FITC conjugated goat anti-mouse IgG (H+L) (Invitrogen). DAPI was used to stain the nucleus. Stained cells were mounted in Moviol, and images were recorded using a 63XNA objective on a Leica inverted microscope.

9—Membrane Extracts

On ice, SW480-Cldn1 cells (˜10⁷/75 cm²) were washed three times with cold PBS and incubated with 1 ml of Tris 10 mM pH7.2 for 30 minutes. Then, cells were scrapped and sonicated 4 times for 5 seconds. Protein extract were centrifugated at 7000 g for 15 minutes and the supernatant was ultracentrifugated at 200 000 g during 15 minutes. The pellet was sonicated and resuspend in PBS. Protein concentration was evaluated by the BCA protein assay reagent (Pierce).

10—Establishment of Three-Dimensional Spheroid

Ultra-low attachment, 96-well round-bottomed plates (Costar) were used to form spheroids. Cells were plated at a density of 5×10⁴ (SW480, SW480-CLDN1, SW620) or 2×10⁴ cells/well (HuH-7). Cells aggregated and merged into three-dimensional (3D) balls with a spheroid configuration within 24 to 48 h. Images of wells were taken with a phase-contrast microscope using a 10 or 5 objective.

11—Assay for Tumor Cell Migration in Boyden Chamber

Cell migration was studied in a Boyden chamber. Briefly, cells were trypsinated, washed 3 times with serum free medium and 50 000 (IGROV, BXPC3) or 100 000 cells (Caco2, HuH-7) were added into the transwell inserts with 8 μm pore (BD Falcon® HTS Fluoroblok™ Inserts). The lower well of the chamber was filled with medium supplemented with 10% FCS. After 21 h incubation, migrated cells were stained with 4 μg/ml of calcein (Sigma-Aldrich 17783-AM) for 1 hour. The number of fluorescent migrated cells was counted in 12 different fields using ImageJ sowftware.

12—Radiolabeling and SPECT-CT Imaging

¹²⁵I was obtained from Perkin Elmer, and 6F6C3 mAb was radiolabeled at the specific activity of 370 MBq/mg for SPECT imaging, using the IODO-GEN (Pierce Chemical Co.) method as previously described (Santoro et al., 2009). All animal experiments were performed in compliance with the guidelines of the French government and the standards of Institut National de la Santé et de la Recherche Médicale for experimental animal studies (agreement CEEA-LR-12052).

Nude mice, 6-8-week-old female athymic nude mice were purchased from Harlan (Gannat, France) and were acclimated for 1 wk before experimental use. They were housed at 22° C. and 55% humidity with a light-dark cycle of 12 h. Food and water were available ad libitum. The mice were force-fed with Lugol solution the day before imaging, and stable iodine was added to drinking water for the entire experimental period.

SPECT-CT imaging: Whole-body SPECT/CT images were acquired at various times (48, 72 and 96 h) after tail vein injection of 16 MBq/50 microgram radiolabeled ¹²⁵I-6F6C3 mAb. Mice were anesthetized with 2% isoflurane and positioned on the bed of 4-head multiplexing multipinhole NanoSPECT camera (Bioscan Inc., Washington, USA). Energy window was centered at 28 keV with ±20% width, acquisition times were defined to obtain 30 000 counts for each projection with 24 projections. Images and maximum intensity projections (MIPs) were reconstructed using the dedicated software Invivoscope® (Bioscan, Inc., Washington, USA) and Mediso InterViewXP® (Mediso, Budapest Hungary). Concurrent microCT whole-body images were performed for anatomic coregistration with SPECT data. Reconstructed data from SPECT and CT were visualized and coregistered using Invivoscope®.

13—Intrasplenic Hepatic Colonization Model

Twenty 6-8-week-old female athymic nude mice were injected in the spleen with 2 millions of SW620-LUC cells (Luciferase-expressing SW620 cells). The spleen was removed after cell injection. On day 1, mice were randomly divided into two groups of 10 mice each. One groupe received intra-peritoneal injection of 6F6C3mAb at 15 mg/kg, the second group received only the vehicle 0.9% NaCl. Treatment with 6F6C3mAb consisted of 3 injections at 15 mg/kg per week. Once weekly, to evaluate metastatic formation and dissemination, luciferase expression was monitored by luminescence imaging after injection of luciferin. At 5 weeks from surgery, mice were sacrificed and the number and the size of metastases on the liver surface were documented.

Results:

1—CLDN1 Expression in Colon Tissues

TMA from 45 colorectal cancer patients including for each patient, normal mucosa, adenoma and adenocarcinoma samples were used to determine CLDN1 expression. We showed a statistically significant increase of the CLDN1 staining from normal mucosa to adenoma (p<0.001), to adenocarcinoma (p<0.001) and from adenoma to adenocarcinoma (p=0.047 or p=0.001 for labelled cells or intensity respectively) (FIG. 1). This result was the same whatever the criterion evaluated: % of labelled cells (FIG. 1A) or mean of labelling intensity (FIG. 1B). CLDN1 immunohistochemistry signals were seen in the membrane as well as in the cytoplasm of tumors cells. The CLDN1-staining was found exclusively in the cytoplasm in normal mucosa (39/45 patients) and in half of adenomas (18/45) while in the second half of adenomas (25/45) and adenocarcinomas (36/45) we observed both membrane and cytoplasmic staining. Furthermore 9% of adenocarcinomas (4/45) displayed an exclusive membrane staining (FIG. 1C). These results show increased expression of CLDN1 in colon cancers together with a change of location.

2—Selection of mAbs Against Human CLDN1

For the selection of mAbs against CLDN1, we generated SW480-CLDN1 cells (SW480 which had acquired CLDN1 expression following stable transfection with the full-length CLDN1 cDNA) and used as a positive target. Mabs screening was performed by FACs experiments using SW480 as negative control. A confirmation screening was performed on SW620 and SW620-shCLDN1 cells. All these lines were first checked for CLDN1 expression by Western Blot. On the basis of this screening we selected three hybridomas secreting mAbs against CLDN1. After subsequent cloning by limiting dilution, we obtained three monoclonal antibodies (mAb) named 6F6C3, 14B7D4 and 15E7B10 (FIG. 2B). Antibody isotyping revealed that 6F6C3 was an IgG3k, and 14B7D4 and 15E7B10 were IgM.

3—Analysis of the Reactivity and Specificity of 6F6C3 mAb

Specificity of 6F6C3 mAb was analyzed by flow cytometry (FACS) using colorectal cell lines with differential CLDN1 expression. Western-blotting experiments were performed using total cell lysates from colorectal cancer cell lines, including SW480, SW480-CLDN1, SW620, SW620shLUC, SW620-shCLDN1, HCT116, LS174T and Caco2. Using commercial anti-CLDN1 antibody, we first evaluated the total expression of CLDN1 in the cell lines (FIG. 3A, 3B) and showed that four cell lines expressed CLDN1 (SW480-CLDN1, SW620, SW620shLUC and Caco2) while four displayed few or no CLDN1 expression (SW480, SW620-shCLDN1, LS174T HCT116). Then we tested by FACS the binding of 6F6C3 mAb on these cell lines (FIG. 3C, 3D). 6F6C3 mAb reacted only with the colon cancer cell lines expressing CLDN1. Furthermore, 6F6C3 mAb did not react with the parental SW480 cells but its reactivity was strongly increased with SW480-CLDN1. Conversely, reactivity of 6F6C3 mAb with SW620 colorectal cells was reduced by at least 85% when CLDN1 expression was knocked down by transduction with CLDN1-specific shRNA.

To further evaluate binding of 6F6C3 mAb to CLDN1, cell lysates were prepared from SW480-CLDN1 and SW480 and then subjected to immunoprecipitation analysis. As a result, 6F6C3 mAb specifically precipitated CLDN1 only on SW480-CLDN1 lysates (FIG. 3E).

By Immunofluorescence study we showed that 6F6C3 mAb is able to bind the surface of the non-impermeabilized SW480-CLDN1 but not SW480 cells (FIG. 3F). Altogether these results suggest that 6F6C3 mAb is specific for CLDN1.

Finally, CLDN1 binding of 6F6C3 was confirmed by BIACORE analysis (FIG. 3G). BIACORE analysis has been performed using the interaction facilities located at the Cancer Research Institute Montpellier (PP2I platform, M. Pugnières). The interaction CLDN1 and 6F6C3 mAb was determined by surface plasmon resonance using BIACORE 3000 instrument (GE Healthcare, Uppsala, Sweden). CLDN1-membrane extracts were immobilised on HPA sensor chip surface. Specific interactions were seen only with 6F6C3 mAb and no with the irrelevant antibody (FIG. 3G).

We have also tested other cancer cell lines (ovarian, pancreatic, breast and prostate) for the CLDN1 expression. We first evaluated by Western blotting the total CLDN1 expression (FIG. 4A) using commercial anti-CLDN1 antibody. Thus we tested reactivity of 6F6C3 mAb by FACs. The four cell lines (BXPC3, PANC-1, SKOV-3 and IGROV-1) overexpressing CLDN1 were recognized by 6F6C3 mAb whereas any reactivity was seen with CLDN1-negative cell lines (FIG. 4B) These results confirmed the human-CLDN1 specificity of 6F6C3 mAb. In addition these cell lines can be used to test biological effect of 6F6C3 mAb on other cancer types.

4—Analysis of Cross-Reactivity with Other CLDNs

After transitory tranfection on SW480 cells of human cDNA clone of CLDN8 (sc320974, Origene technologies, USA) and murine cDNA clone of CLDN1 (IRAVp968A105D, LifeScience), we analysed by FACs cross-reactivity of 6F6C3 mAb. As shown in FIG. 5A, all the transfections showed an overexpression of the transfected CLDN and as already described, SW480 expressed CLDN7 as well as CLDN3 and CLDN4 (Dhawan et al., 2011). 6F6C3 mAb did not react neither with SW480-mCLDN1 nor with SW480-CLDN8 and nor with SW480 (FIG. 5B). These results indicate that 6F6C3 mAb did not recognize CLDN8, CLDN7 and probably CLDN3 and CLDN4. Furthermore 6F6C3 did not cross-react with murine CLDN1 which has 94% and 92% of identity at extracellular level with human CLDN1 (Table 2).

TABLE 2
Percentage of identity between extracellular
domains of CLDNs (ClustalW2)

| CLDNs | ECL1^{a} | ECL2 |
|---|---|---|
| murineCLDN1 | 94% | 92% |
| CLDN8 | 50% | 29% |
| CLDN7 | 69% | 51% |
| CLDN3 | 60% | 33% |
| CLDN4 | 62% | 29% |

^{a}ECL = extracellular loop

5—In Vitro Biologic Effects of mAb6F6C3

Survival.

The effect of 6F6C3 mAb on survival of cells was tested by a clonogenic assay which is based on the ability of a single cell to grow into a colony (Franken et al., 2006). Treatment by 6F6C3mAb reduced the number of colony-forming cells for Caco2 colorectal cancer cells (FIG. 6A). This reduction was concentration dependent as it was of 37% for 6F6C3mAb at 50 μg/ml and of 68% for 100 μg/ml (FIG. 6B). In order to confirm that the observed effect is specific of CLD1, we performed clonogenic assay on six other cell lines overexpressing CLDN1 (BXPC3, PANC-1, SKOV-3, IGROV-1, HuH-7 and SW620) and on SW480 as negative control. As shown in FIG. 6C, 6F6C3mAb was able to inhibit the formation of colonies for all the cell lines excepted for the CLDN1-negative cell line SW480, indicating that this effect is specific of the CLDN1 binding by 6F6C3mAb.

Growth.

The effect 6F6C3 mAb on cell growth was studied on 3D culture. The shape of 3D spheres varied depending on cell line used. SW480 and SW480-CLDN1 formed single, tight, spherical and regular spheroids, HuH-7 too but less regular and accompanied by micro spheroids while SW620 formed aggregates (FIG. 7). When the cells were incubated with 6F6C3mAb, we observed a decrease of sphere size compared to the non-treated cells or cells treated with an irrelevant mAb for the three CLDN1-positive cell lines (FIG. 7). Any effect was shown on CLD1-negative cell line, SW480. These results demonstrated that 6F6C3mAb influence the cellular growth of CLDN1-positive cell lines.

Migration.

The effect 6F6C3mAb on migration was measured by a Boyden Chamber assay. Cells were treated by 6F6C3mAb or an irrelevant mAb. The results showed (FIG. 8) that 6F6C3mAb was be able to significantly affect the migration of all the CLDN1-positive cell lines tested.

Altogether, the binding of 6F6C3mAb on membrane CLDN1 affects growth and survival of CLDN1-positive cell lines as well as their migration capacity.

6—Biodistribution

To determine the tumor uptake and the ability of 6F6C3 mAb to specifically target CLDN1 in vivo we performed small-animal SPECT/CT study (single-photon emission computed tomography) (M. Busson, Plate-forme Imagerie du Petit Animal par Bioluminescence et Scintigraphie, IRCM). Two female athymic nude mice were grafted subcutaneously by injecting SW480-CLDN1 (3.10⁶) cells into the right flank and SW480 into the left flank. Intravenous injection of 50 μg (500 μCi) of ¹²⁵I labelled-6F6C3mAb was performed once the tumor reached 100 mm³. Then CT and SPECT scans were acquired 48 h, 72 h and 96 h after injection. At 48 h, we observed a strong localisation of ¹²⁵I labelled-6F6C3mAb in the SW480-CLDN1-grafted tumor and in stomach and in ladder but not in the SW480-grafted tumor. SPECT/CT imaging 72 hours after injection showed high and specific uptake of ¹²⁵I labelled-6F6C3mAb only in the SW480-CLDN1-grafted tumor (FIG. 9). This result confirms in vivo the specificity of 6F6C3mAb to human CLDN1.

7—In Vivo Tumour Growth Inhibition Study:

All in vivo experiments were performed in compliance with the French guidelines for experimental animal studies (Agreement CEEA-LR-12053). Nude mice, 6-8-week-old female athymic nude mice were purchased from Harlan (Gannat, France).

SW620 (3.10⁶) cells were suspended in culture medium and were injected subcutaneously (s.c.) into the right flank of athymic nude mice. Tumour-bearing mice were randomized in the different groups when the tumours reached approximately the same volume (100 mm3). The mice were treated by intra-peritoneal injections (i.p.) with 0.9% NaCl or mAb6F6C3. The amounts of injected mAb were 15 mg/Kg per injection, twice a week for three weeks consecutively for the first experiment and 3 injections per week at 15 mg/Kg for the second one.

Tumour dimensions were measured bi-weekly with a caliper and the volumes calculated by the formula: D1×D2×D3/2.

The results were expressed by tumor growth kinetics of xenografted mice (FIG. 10A) and showed that mAb6F6C3 treated-groups had a significant (p=0,018) reduced growth compared to the control group. In addition this effect was dose-dependent as we observed a significant growth difference (p=0.011) between first and second experiment.

An adapted Kaplan-Meier survival curve, using the time taken for the tumour to reach a determined volume of 1500 mm³ (FIG. 10B), showed that the median delay is 7 days longer for the treated group as compared with the control NaCl group. (A median delay was defined as the time at which 50% of the mice had a tumour reaching the determined volume)

8—Intrasplenic Hepatic Colonization

To evaluate the effects of mAb6F6C3 on the formation of metastases in liver, SW620-LUC cells were injected in mice through the intrasplenic/portal route. Mice were treated or not with 6F6C3mAb. At the experimental endpoint, livers were examined and number of metastases was determined in both groups. Consistent with previous report (Dhawan et al., 2005) SW620 metastasized to the liver. In control group, livers were shown to be invaded at higher rate compared to the treated group (FIG. 11A). Indeed, the median of number of metastases was increased in control group (FIG. 11B). Furthermore, in the control group all the mice had metastases with 50% having more than 10 liver metastases while 30% of treated group mice had no metastasis or only a micro-metastasis (FIG. 11C).

9—Sequence of mAb6F6C3:

We have cloned and characterized the variable domain of the light and heavy chains of said 6F6C3 mAb, and thus determined the complementarity determining regions (CDRs) of said antibody. The monoclonal antibody is an immunoglobulin of the IgG3 heavy chain and kappa light chain (Table 1 supra).

### REFERENCES

Throughout this application, various references describe the state of the art to which this invention pertains. The disclosures of these references are hereby incorporated by reference into the present disclosure.
Aro K, Rosa L E, Bello I O, Soini Y, Mäkitie A A, Salo T, Leivo I. (2011) Expression pattern of claudins 1 and 3-an auxiliary tool in predicting behavior of mucoepidermoid carcinoma of salivary gland origin. Virchows Arch. 458:341-8.
Bénard J, Da Silva J, De Blois M C, Boyer P, Duvillard P, Chiric E, Riou G. (1985) Characterization of a human ovarian adenocarcinoma line, IGROV1, in tissue culture and in nude mice. Cancer Res. 45:4970-9.
Chao, Y.-C., Pan, S.-H., Yang, S.-C., Yu, S.-L., Che, T.-F., Lin, C.-W., Tsai, M.-S., Chang, G.-C., Wu, C.-H., Wu, Y.-Y., et al. (2009). Claudin-1 Is a Metastasis Suppressor and Correlates with Clinical Outcome in Lung Adenocarcinoma. Am. J. Respir. Crit. Care Med. 179, 123-133.
de Aquino A R, de Carvalho C H, Nonaka C F, Freitas Rde A, de Souza L B, Pinto L P. (2012) Immunoexpression of claudin-1 and Nm23-H1 in metastatic and nonmetastatic lower lip squamous-cell carcinoma. Appl Immunohistochem Mol Morphol. 20:595-601.
De Oliveira, S. S., De Oliveira, I. M., De Souza, W., and Morgado-Díaz, J. A. (2005). Claudins upregulation in human colorectal cancer. FEBS Lett 579, 6179-6185.
Dhawan, P., Ahmad, R., Chaturvedi, R., Smith, J. J., Midha, R., Mittal, M. K., Krishnan, M., Chen, X., Eschrich, S., Yeatman, T. J., et al. (2011). Claudin-2 expression increases tumorigenicity of colon cancer cells: role of epidermal growth factor receptor activation. Oncogene 30, 3234-3247.
Dhawan, P., Singh, A. B., Deane, N. G., No, Y., Shiou, S.-R., Schmidt, C., Neff, J., Washington, M. K., and Beauchamp, R. D. (2005). Claudin-1 regulates cellular transformation and metastatic behavior in colon cancer. J Clin Invest 115, 1765-1776.
Dos Reis, P. P., Bharadwaj, R. R., Machado, J., Macmillan, C., Pintilie, M., Sukhai, M. A., Perez-Ordonez, B., Gullane, P., Irish, J., and Kamel-Reid, S. (2008). Claudin 1 overexpression increases invasion and is associated with aggressive histological features in oral squamous cell carcinoma. Cancer 113, 3169-3180.
English D P, Santin A D. Claudins Overexpression in Ovarian Cancer: Potential Targets for Clostridium Perfringens Enterotoxin (CPE) Based Diagnosis and Therapy. (2013) Int J Mol Sci. 17:10412-37.
Fèvre Montange M, Vasiljevic A, Bergemer Fouquet A M, Bernier M, Champier J, Chrétien F, Figarella-Branger D, Kemeny J L, Lechapt-Zalcman E, Michalak S, Miguel C, Mokthari K, Pommepuy I, Quintin Roué I, Rousseau A, Saint-Pierre G, Salon C, Uro-Coste E, Varlet P, Kratzer I, Ghersi-Egea J F, Jouvet A. (2012) Histopathologic and ultrastructural features and claudin expression in papillary tumors of the pineal region: a multicenter analysis. Am J Surg Pathol. 36:916-28.
Franken, N. A. P., Rodermond, H. M., Stap, J., Haveman, J., and Van Bree, C. (2006). Clonogenic assay of cells in vitro. Nat Protoc 1, 2315-2319.
Furuse, M., Fujita, K., Hiiragi, T., Fujimoto, K., and Tsukita, S. (1998). Claudin-1 and -2: novel integral membrane proteins localizing at tight junctions with no sequence similarity to occludin. J. Cell Biol. 141, 1539-1550.
Granci, V., Bibeau, F., Kramar, A., Boissière-Michot, F., Thézénas, S., Thirion, A., Gongora, C., Martineau, P., Del Rio, M., and Ychou, M. (2008). Prognostic significance of TRAIL-R1 and TRAIL-R3 expression in metastatic colorectal carcinomas. Eur. J. Cancer 44, 2312-2318.
Gröne, J., Weber, B., Staub, E., Heinze, M., Klaman, I., Pilarsky, C., Hermann, K., Castanos-Velez, E., Röpcke, S., Mann, B., et al. (2007). Differential expression of genes encoding tight junction proteins in colorectal cancer: frequent dysregulation of claudin-1, -8 and -12. Int J Colorectal Dis 22, 651-659.
Hammarstrom, S., Shively, J. E., Paxton, R. J., Beatty, B. G., Larsson, A., Ghosh, R., Bormer, O., Buchegger, F., Mach, J. P., and Burtin, P. (1989). Antigenic sites in carcinoembryonic antigen. Cancer Res. 49, 4852-4858.
Haskell, C. M., Buchegger, F., Schreyer, M., Carrel, S., and Mach, J. P. (1983). Monoclonal antibodies to carcinoembryonic antigen: ionic strength as a factor in the selection of antibodies for immuno scintigraphy. Cancer Res. 43, 3857-3864.
Huo, Q., Kinugasa, T., Wang, L., Huang, J., Zhao, J., Shibaguchi, H., Kuroki, M., Tanaka, T., Yamashita, Y., Nabeshima, K., et al. (2009). Claudin-1 protein is a major factor involved in the tumorigenesis of colorectal cancer. Anticancer Res 29, 851-857.
Hsueh C, Chang Y S, Tseng N M, Liao C T, Hsueh S, Chang J H, Wu I C, Chang K P. (2010) Expression pattern and prognostic significance of claudins 1, 4, and 7 in nasopharyngeal carcinoma. Hum Pathol. 41:944-50.
Kinugasa, T., Akagi, Y., Yoshida, T., Ryu, Y., Shiratuchi, I., Ishibashi, N., and Shirouzu, K. (2010). Increased claudin-1 protein expression contributes to tumorigenesis in ulcerative colitis-associated colorectal cancer. Anticancer Res. 30, 3181-3186.
Kinugasa, T., Huo, Q., Higashi, D., Shibaguchi, H., Kuroki, M., Tanaka, T., Futami, K., Yamashita, Y., Hachimine, K., Maekawa, S., et al. (2007). Selective up-regulation of claudin-1 and claudin-2 in colorectal cancer. Anticancer Res 27, 3729-3734.
Kominsky, S. L. (2006). Claudins: emerging targets for cancer therapy. Expert Rev Mol Med 8, 1-11.
Kondoh A, Takano K, Kojima T, Ohkuni T, Kamekura R, Ogasawara N, Go M, Sawada N, Himi T. (2011) Altered expression of claudin-1, claudin-7, and tricellulin regardless of human papilloma virus infection in human tonsillar squamous cell carcinoma. Acta Otolaryngol. 2011 131:861-8
Krause, G., Winkler, L., Mueller, S. L., Haseloff, R. F., Piontek, J., and Blasig, I. E. (2008). Structure and function of claudins. Biochimica Et Biophysica Acta (BBA)—Biomembranes 1778, 631-645.
Lal-Nag, M., and Morin, P. J. (2009). The claudins. Genome Biol 10, 235-235.
Leotlela, P. D., Wade, M. S., Duray, P. H., Rhode, M. J., Brown, H. F., Rosenthal, D. T., Dissanayake, S. K., Earley, R., Indig, F. E., Nickoloff, B. J., et al. (2007). Claudin-1 overexpression in melanoma is regulated by PKC and contributes to melanoma cell motility. Oncogene 26, 3846-3856.
Liu T, Cheng W, Lai D, Huang Y, Guo L. (2010) Characterization of primary ovarian cancer cells in different culture systems. Oncol Rep. 23:1277-84.
Lu, S., Singh, K., Mangray, S., Tavares, R., Noble, L., Resnick, M. B., and Yakirevich, E. (2012). Claudin expression in high-grade invasive ductal carcinoma of the breast: correlation with the molecular subtype. Modern Pathology.
Miwa, N., Furuse, M., Tsukita, S., Niikawa, N., Nakamura, Y., and Furukawa, Y. (2001). Involvement of claudin-1 in the beta-catenin/Tcf signaling pathway and its frequent upregulation in human colorectal cancers. Oncol. Res 12, 469-476.
Myal Y, Leygue E, Blanchard A A. (2010) Claudin 1 in breast tumorigenesis: revelation of a possible novel “claudin high” subset of breast cancers. J Biomed Biotechnol 2010:956897.
Nakanishi K, Ogata S, Hiroi S, Tominaga S, Aida S, Kawai T. (2008) Expression of occludin and claudins 1, 3, 4, and 7 in urothelial carcinoma of the upper urinary tract. Am J Clin Pathol. 130:43-9.
Németh, J., Németh, Z., Tátrai, P., Péter, I., Somorácz, A., Szász, A. M., Kiss, A., and Schaff, Z. (2010). High expression of claudin-1 protein in papillary thyroid tumor and its regional lymph node metastasis. Pathol. Oncol. Res. 16, 19-27.
Olive, M., Untawale, S., Coffey, R. J., Siciliano, M. J., Wildrick, D. M., Fritsche, H., Pathak, S., Cherry, L. M., Blick, M., and Lointier, P. (1993). Characterization of the DiFi rectal carcinoma cell line derived from a familial adenomatous polyposis patient. In Vitro Cell. Dev. Biol. 29A, 239-248.
Ouban A, Hamdan H, Hakam A, Ahmed A A. (2012) Claudin-1 expression in squamous cell carcinomas of different organs: comparative study of cancerous tissues and normal controls. Int J Surg Pathol. 20:132-8.
Santoro, L., Boutaleb, S., Garambois, V., Bascoul-Mollevi, C., Boudousq, V., Kotzki, P.-O., Pèlegrin, M., Navarro-Teulon, I., Pèlegrin, A., and Pouget, J.-P. (2009). Noninternalizing monoclonal antibodies are suitable candidates for 125I radioimmunotherapy of small-volume peritoneal carcinomatosis. J. Nucl. Med. 50, 2033-2041.
Shin H I, Kim B H, Chang H S, Kim C I, Jung H R, Park C H. (2011) Expression of claudin-1 and -7 in clear cell renal cell carcinoma and its clinical significance. Korean J Urol. 52:317-22.
Singh, A. B., Sharma, A., and Dhawan, P. (2010). Claudin family of proteins and cancer: an overview. J Oncol 2010, 541957.
Soini Y, Kinnula V, Kahlos K, Pääkkö P. (2006) Claudins in differential diagnosis between mesothelioma and metastatic adenocarcinoma of the pleura. J Clin Pathol. 59:250-4.
Swisshelm, K., Machl, A., Planitzer, S., Robertson, R., Kubbies, M., and Hosier, S. (1999). SEMP1, a senescence-associated cDNA isolated from human mammary epithelial cells, is a member of an epithelial membrane protein superfamily. Gene 226, 285-295.
Szabó, I., Kiss, A., Schaff, Z., and Sobel, G. (2009). Claudins as diagnostic and prognostic markers in gynecological cancer. Histol. Histopathol 24, 1607-1615.
Szász, A. M., Nyirády, P., Majoros, A., Szendrõi, A., Szûcs, M., Székely, E., Tõkés, A.-M., Romics, I., and Kulka, J. (2009). beta-catenin expression and claudin expression pattern as prognostic factors of prostatic cancer progression. BJU Int.
Takala H, Saarnio J, Wiik H, Soini Y. (2007) Claudins 1, 3, 4, 5 and 7 in esophageal cancer: loss of claudin 3 and 4 expression is associated with metastaticbehavior. APMIS. 115:838-47.
Tsukahara M, Nagai H, Kamiakito T, Kawata H, Takayashiki N, Saito K, Tanaka A. (2005) Distinct expression patterns of claudin-1 and claudin-4 in intraductal papillary-mucinous tumors of the pancreas. Pathol Int. 55:63-9.
Tsukita, S., and Furuse, M. (2006). The Structure and Function of Claudins, Cell Adhesion Molecules at Tight Junctions Annals of the New York Academy of Sciences 915, 129-135.
Tsukita, S., Furuse, M., and Itoh, M. (2001). Multifunctional strands in tight junctions. Nat. Rev. Mol. Cell Biol. 2, 285-293.
Weber C R, Nalle S C, Tretiakova M, Rubin D T, Turner J R. (2008) Claudin-1 and claudin-2 expression is elevated in inflammatory bowel disease and may contribute to early neoplastic transformation. Lab Invest. 88:1110-20.
Wu, Y.-L., Zhang, S., Wang, G.-R., and Chen, Y.-P. (2008). Expression transformation of claudin-1 in the process of gastric adenocarcinoma invasion. World J. Gastroenterol. 14, 4943-4948.
Yoon, C.-H., Kim, M.-J., Park, M.-J., Park, I.-C., Hwang, S.-G., An, S., Choi, Y.-H., Yoon, G., and Lee, S.-J. (2009). Claudin-1 acts through c-abl-pkcdelta signaling and has a causal role in the acquisition of invasive capacity in human liver cells. J. Biol. Chem.

1. An anti-Claudin 1 (CLDN1) antibody comprising a heavy chain variable region comprising SEQ ID NO:2 in the H-CDR1 region, SEQ ID NO:3 in the H-CDR2 region and SEQ ID NO:4 in the H-CDR3 region; and a light chain variable region comprising SEQ ID NO:6 in the L-CDR1 region, SEQ ID NO:7 in the L-CDR2 region and SEQ ID NO:8 in the L-CDR3 region.

2. The anti-claudin 1 antibody of claim 1 wherein the heavy chain variable region of said antibody has the amino acid sequence set forth as SEQ ID NO: 1.

3. The anti-claudin 1 antibody of claim 1 wherein the light chain variable region has the amino acid sequence set forth as SEQ ID NO: 5.

4. The anti-claudin 1 antibody of claim 1 wherein the heavy chain variable region of said antibody has the amino acid sequence set forth as SEQ ID NO: 1 or 3 and the light chain variable region has the amino acid sequence set forth as SEQ ID NO: 5.

5. The anti-claudin 1 antibody of claim 1 which is a chimeric antibody.

6. The anti-claudin 1 antibody of claim 1 which is a humanized antibody.

7. A fragment of an antibody of claim 1 which is selected from the group consisting of Fv, Fab, F(ab′)2, Fab′, dsFv, scFv, sc(Fv)2 and a diabody.

8. An anti-Claudin 1 (CLDN1) antibody comprising a heavy chain wherein the variable domain comprises:
a H-CDR1 having at least 90% or 95% identity with sequence set forth as SEQ ID NO: 2,
a H-CDR2 having at least 90% or 95% identity with sequence set forth as SEQ ID NO: 3,
a H-CDR3 having at least 90% or 95% identity with sequence set forth as SEQ ID NO: 4,
a L-CDR1 having at least 90% or 95% identity with sequence set forth as SEQ ID NO: 6,
a L-CDR2 having at least 90% or 95% identity with sequence set forth as SEQ ID NO: 7,
a L-CDR3 having at least 90% or 95% identity with sequence set forth as SEQ ID NO: 8, and
wherein the anti-CLDN1 antibody specifically binds to CLDN1 with substantially the same affinity as an antibody comprising a heavy chain wherein the variable domain comprises SEQ ID NO: 2 for H-CDR1, SEQ ID NO: 3 for H-CDR2 and SEQ ID NO: 4 for H-CDR3 and a light chain wherein the variable domain comprises SEQ ID NO: 6 for L-CDR1, SEQ ID NO: 7 for L-CDR2 and SEQ ID NO: 8 for L-CDR3.

9. A polypeptide which has a sequence selected from the group consisting of SEQ ID NO: 1, SEQ ID NO:2, SEQ ID NO:3, SEQ ID NO:4, SEQ ID NO:5;
SEQ ID NO: 6; SEQ ID NO:7 and SEQ ID NO:8.

10. A nucleic acid sequence encoding the antibody or fragment according to claim 1.

11. A vector comprising the nucleic acid of claim 10.

12. A host cell which has been transfected, infected or transformed by the nucleic acid of claim 10 or the vector of claim 11.

13. The antibody according to claim 1 which is conjugated to a detectable label to form an anti-CLDN1 immunoconjugate.

14. The antibody according to claim 1 which is conjugated to a therapeutic agent.

15. The antibody of claim 14 wherein the therapeutic agent is selected from the group consisting of chemotherapeutic agents, prodrug converting enzymes, radioactive isotopes or compounds, and toxins.

16. A method of diagnosing a disease associated with CLDN1 overexpression comprising the steps of (a) contacting a biological sample of a subject likely to suffer from a disease associated with CLDN1 overexpression with an antibody according to claim 1 in conditions sufficient for the antibody to form complexes with cells of the biological sample that express CLDN1; and (b) detecting and/or quantifying said complexes, whereby the detection of said complexes is indicative of a disease associated with CLDN1 overexpression.

17. The method according to claim 16 wherein the disease is a cancer.

18. A method of treating a cancer in a subject in need thereof comprising administering the subject with a therapeutically effective amount of an antibody according to claim 1.

19. The method of claim 18 wherein the cancer is selected from the group consisting of colorectal cancer, gynaecological cancers, ovarian cancers, cervical neoplasias, melanoma, squamous cell carcinoma such as oral SCC, lower lip SCC, head and neck, skin SCC, Tonsillar SCC, gastric adenocarcinoma, thyroid carcinoma, mammary carcinoma, Neuroepithelial papillary tumor of the pineal region (PTPR), clear cell renal cell carcinoma, mucoepidermoid carcinoma (MEC) of salivary gland, nasopharyngeal carcinoma, urothelial carcinoma of the upper urinary tract, esophageal carcinoma, mesotheliomas, pleural metastatic adenocarcinoma, and pancreas tumors.

20. A method of treating a HCV infection in a subject in need thereof comprising administering the subject with a therapeutically effective amount of an antibody according claim 1.

21. A pharmaceutical composition comprising an antibody according to claim 1.

22. A kit comprising an antibody according to claim 1.

23. The anti-claudin 1 antibody of claim 5, wherein said chimeric antibody is a chimeric mouse/human antibody.

24. The method according to claim 17 wherein the cancer is colorectal cancer.
